(19) United States
(12) Patent Application Publication
(10) Pub. No.: US 2023/0309882 A1
(43) Pub. Date: Oct. 5, 2023

(54) MULTISPECTRAL REALITY DETECTOR SYSTEM

(71) Applicant: Al Seer Pte. Ltd., Singapore (SG)

(72) Inventors: Dennis Mingjie Ye, Singapore (SG); Anmol Dua, Delhi (IN); A M Shahruj Rashid, Singapore (SG); Christopher Lee Asplund, Singapore (SG); Jiamin Bai, Singapore (SG); Natalie Tan, Singapore (SG); Yen Ning Chang, Singapore (SG); Ying Chong Sim, Singapore (SG)

(21) Appl. No.: 17/952,000

(22) Filed: Sep. 23, 2022

Related U.S. Application Data
(60) Provisional application No. 63/327,739, filed on Apr. 5, 2022.

(51) Int. Cl. A61B 5/16; G06V 40/16; G06F 3/01

(52) U.S. Cl. CPC A61B 5/164; G06V 40/174; G06F 3/013

(57) ABSTRACT

Sensor data of a subject is received, wherein the sensor data includes eye tracking data and visible light image data. Using one or more processors, the sensor data is automatically analyzed to determine one or more metrics. Using the one or more metrics, an indicator associated with a likelihood the subject is being deceptive is determined.

### CROSS REFERENCE TO OTHER APPLICATIONS

This application claims priority to U.S. Provisional Patent Application No. 63/327,739 entitled REALITY DETECTOR filed Apr. 5, 2022 which is incorporated herein by reference for all purposes.

### BACKGROUND OF THE INVENTION

The analysis of a subject's responses to questions can be evaluated to predict whether the subject is being truthful. For example, subjects with potential access to sensitive material or in positions that require a certain degree of trust may be evaluated with respect to certain topics, such as their background and activities. As another example, a subject can be questioned with respect to their knowledge of an event to determine how they are associated with the event based on the truthfulness of their responses. Typically, the deception analysis process includes physically attaching invasive devices to the subject in question and an administrator asking a series of questions and evaluating the subject's responses to determine which answers indicate deception.

### BRIEF DESCRIPTION OF THE DRAWINGS

Various embodiments of the invention are disclosed in the following detailed description and the accompanying drawings.

FIG. 1 is a block diagram illustrating an embodiment of a multispectral deception analysis system for assessing deception.

FIG. 2 is a block diagram illustrating sensor components of an embodiment of an interviewee terminal for a multispectral deception analysis system.

FIG. 3 is a block diagram illustrating the arrangement of different sensor and output components an embodiment of an interviewee terminal for a multispectral deception analysis system.

FIG. 4 is a flow chart illustrating an embodiment of a process for assessing deception in a subject.

FIG. 5 is a flow chart illustrating an embodiment of a process for performing an interview to assess the likelihood of deception in a subject's responses.

FIG. 6 is a flow chart illustrating an embodiment of a process for analyzing eye tracking sensor data for determining behavioral cues.

FIG. 7 is a flow chart illustrating an embodiment of a process for analyzing visible image data for determining behavioral cues.

FIG. 8 is a flow chart illustrating an embodiment of a process for analyzing sensor data for determining facial and body expression behavioral cues.

FIG. 9 is a flow chart illustrating an embodiment of a process for analyzing thermal image data for determining behavioral cues.

FIG. 10 is a flow chart illustrating an embodiment of a process for analyzing sensor data for determining a subject's respiration rate.

FIG. 11 is a diagram illustrating an embodiment of a playback user interface for viewing key moments of a subject's interview responses.

FIG. 12 is a diagram illustrating an embodiment of an interviewee user interface.

FIG. 13 is a diagram illustrating an embodiment of an interviewer user interface for viewing records.

FIG. 14 is a diagram illustrating an embodiment of an interactive view session user interface screen for viewing an interview.

FIGS. 15A and 15B are diagrams illustrating an embodiment of an interviewer user interface for performing an interview.

FIGS. 16A-16E are diagrams illustrating an embodiment of an interactive view session user interface screen for viewing an interview.

FIG. 17 is a functional diagram illustrating a programmed computer system for performing deception analysis of an interviewee subject.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

A multispectral truth detector system is disclosed. Using the techniques and systems disclosed herein, a multispectral deception analysis system can predict whether a subject is exhibiting indicators associated with deceptive behavior. For example, an interviewer positions themselves in front of an interviewer terminal and the subject in front of a contactless interviewee terminal. The interviewee terminal is equipped with multiple sensors such as an eye tracking sensor, a visible light sensor, and a microphone. Additional sensors may be incorporated as well, such as a thermal sensor. In various embodiments, the sensors are less invasive and do not require physical contact/attachment on the interviewee, allowing easy and fast setup as well as enabling the interviewer to be remote, optional, and/or automated. The interviewee terminal is equipped with audio/video output to play voice prompts and display questions to the subject. In various embodiments, the interviewer can initiate an interview session with the subject from an optional interviewer terminal. For example, the interview can be automated without the need for an interviewer to monitor and/or participate in the interview. In various embodiments, the interview can be a live or automated interview. For example, using an automated interview, the questions are automatically generated and provided to the interviewer and interviewee. In contrast, a live interview may include automatically generated questions as well as questions initiated by the interviewer. As the interviewee responds to the provided questions during the interview process, the subject's responses are captured. The captured response data includes the subject's verbal answers as well as their behavioral responses. In some embodiments, the subject enters responses to the interview questions using an input device of the interviewee terminal, for example, using a mouse, trackpad, touchscreen, or another input device.

In the disclosed embodiments, the deception analysis system is a multispectral truth detector system and utilizes multiple sensors to capture different data related to a subject's responses. The different captured data is analyzed to determine metrics associated with behavioral cues. For example, an eye tracking camera can capture pupil changes, blink rate, and fixations, among other pupil features. Similarly, a visible light camera can capture visible images of the subject to determine heart rate and/or facial expressions, among other features. In some embodiments, a thermal sensor captures thermal images to determine respiration rate and/or temperature fluctuations, among other features. In some embodiments, the captured data is analyzed using machine learning and/or computer vision techniques to determine one or more response metrics, such as metrics associated with identified behavioral cues. For example, one or more deep learning models can be used to identify different behavioral cues and/or predict the likelihood of deception associated with a detected behavioral cue such as a combination of facial micro expressions.

In various embodiments, the multispectral deception analysis system presents the interviewer with deception assessment results based on deception metrics determined by analyzing the captured sensor data. For example, an interviewer user interface can provide in real time an associated assessment of the subject being interviewed. The provided deception metrics and an associated assessment can be provided as a user interface indicator, such as a user interface component displaying a likelihood of deception, a metric value indicator corresponding to the likelihood of deception, one or more metric values corresponding to detected behavioral cues, and/or another appropriate indicator. For example, an indicator can display in real time the subject's response pause time, respiration rate, blink rate, pupil features, and/or gaze fixation duration, among other metrics. The determined metrics can be associated with responses and/or questions presented to the subject during an interview. In various embodiments, the results can be viewed both in real time as well as post interview. For example, an interview and its deception assessment results can be stored and reviewed at a later date, for example, from a remote terminal or network computing device by accessing a deception analysis service incorporated as part of the deception analysis system.

In some embodiments, eye tracking data of a subject and a visible light image of the subject are received. For example, an eye tracking sensor is used to capture eye tracking data of a subject and an RGB camera is used to capture visible light image data of the subject. The different sensor data can be captured by a contactless interviewee terminal equipped with the appropriate sensors and placed in front of the subject during an interview process. Using one or more processors, the eye tracking data and the visible light image is automatically analyzed to determine one or more metrics. For example, the captured sensor data can be analyzed to determine behavioral cues associated with deception. The eye tracking data can be analyzed to determine pupil features such as pupil changes, gaze direction, and fixation duration, among other eye-tracking pupil features. The visible light image data can be analyzed to determine the heart rate of the subject and/or facial expressions, among other features. In some embodiments, the captured data is analyzed using machine learning and/or computer vision techniques to determine the one or more metrics associated with the sensor data. Using the one or more metrics, an indicator associated with a likelihood the subject is being deceptive is determined. For example, the determined indicator can be a metric value, a user interface component, a deception assessment result, or another appropriate indicator associated with a likelihood the subject is being deceptive. In some embodiments, the determined indicator is a metric value indicator, such as a determined metric value corresponding to a detected behavioral cue. For example, a determined metric value corresponding to a detected behavioral cue can be associated with a determined response pause time, pupil feature, gaze fixation duration, blink rate, heart rate, and respiration rate, among other metric values. In some embodiments, the determined indicator is a user interface component that displays the likelihood of deception or a detected behavioral cue metric.

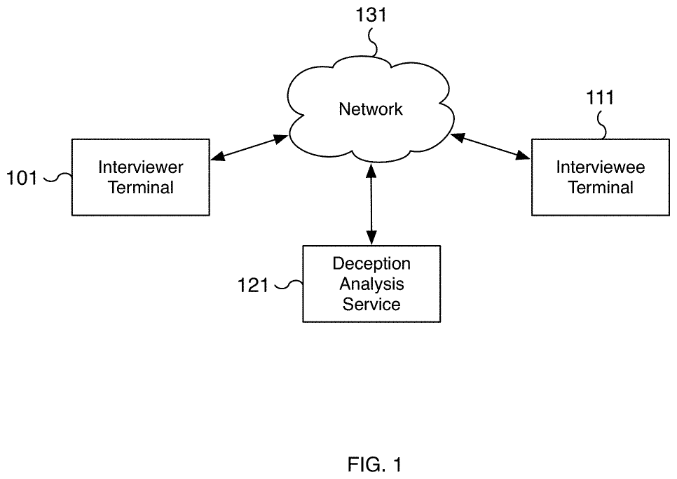

FIG. 1 is a block diagram illustrating an embodiment of a multispectral deception analysis system for assessing deception. In the example shown, the multispectral deception analysis system includes interviewer terminal 101, interviewee terminal 111, deception analysis service 121, and network 131. Using the multispectral deception analysis system of FIG. 1, an interviewer positioned at interviewer terminal 101 can initiate an interview with a subject positioned at interviewee terminal 111. The interview can be a live or automated interview and is analyzed for indicators of deception. In various embodiments, the deception analysis is performed by deception analysis service 121. As shown in FIG. 1, interviewer terminal 101, interviewee terminal 111, and deception analysis service 121 are communicatively connected via network 131. In some embodiments, network 131 is the Internet. Interviewer terminal 101 and interviewee terminal 111 can be positioned in the same location, such as in the same room on opposite sides of a table, or remote from one another, such as in separate rooms or buildings.

In some embodiments, interviewer terminal 101 is a network computing device that allows an interviewer to access deception analysis service 121. Using interviewer terminal 101, an interviewer can initiate an interview with a subject and is provided with a deception assessment of the subject's responses. In various embodiments, interviewer terminal 101 includes at least an interactive display for displaying and allowing the interviewer to interact with the real-time deception analysis of the subject being interviewed. In some embodiments, interviewer terminal 101 includes a video and/or audio feed of the subject during the interview and overlayed user interface components annotating determined behavioral cues and their impact on the provided deception analysis results. For example, using interviewer terminal 101, an interviewer can monitor a subject's response pause time, blink rate, pupil features, gaze fixation duration, gaze target, heart rate, respiration rate, facial expressions including micro expressions, and/or temperature fluctuations, among other detected behavioral cues along with a video/audio feed of the subject as they respond to questions. In some embodiments, interviewer terminal 101 also presents the current question as well as a visual display of the subject's answer. In the event the subject's response is a verbal response, a transcript of the response can be included. In addition to its use in initiating and performing interviews, interviewer terminal 101 can be used to retrieve previously performed interviews. For example, using interviewer terminal 101, a previously performed interview and its corresponding analysis can be retrieved, viewed, and annotated by a user or operator of interviewer terminal 101.

In some embodiments, interviewee terminal 111 is a network computing device used to initiate and direct a subject during an interview and to capture the subject's corresponding responses. For example, a subject is positioned at interviewee terminal 111 and is provided with a sequence of questions in visual and/or audio format. In some embodiments, a display of interviewee terminal 111 can display a sequence of questions for the subject to answer. The subject's response to the questions are captured by the sensors of interviewee terminal 111. The captured responses include both the subject's answer, such as a verbal or an interactive answer provided using an input device such as a mouse, as well as the subject's behavioral responses. In various embodiments, interviewee terminal 111 includes multiple sensors, such as an eye tracking camera, an RGB camera, a thermal camera, microphone, a mouse, a touchpad, and/or a touch screen, for capturing different aspects of the subject's response.

As shown in FIG. 1, interviewee terminal 111 is communicatively connected to interviewer terminal 101 and/or deception analysis service 121. For example, the questions directed to the subject can be provided to the subject at interviewee terminal 111 by an interviewer positioned at interviewer terminal 101 and/or generated and provided by deception analysis service 121. In various embodiments, the sensor data captured by interviewee terminal 111 is transmitted to interviewer terminal 101 and/or deception analysis service 121. For example, the sensor data can be transmitted to deception analysis service 121 where deception analysis is performed. The analysis results can then be provided from deception analysis service 121 to interviewer terminal 101. In some embodiments, interviewer terminal 101 and/or interviewee terminal 111 include one or more processors for performing at least a portion of the deception analysis locally at interviewer terminal 101 and/or interviewee terminal 111. In various embodiments, interviewee terminal 111 is a contactless interviewee terminal that does not require physically attaching sensor devices to the subject.

In some embodiments, deception analysis service 121 is a service that provides deception analysis results based on a subject's responses to interview questions. Using the service provided by deception analysis service 121, a subject can participate in an interview where the responses are analyzed to provide a deception assessment. The conducted interview can be a live or fully automated interview. For some interviews, some or all of the questions are automatically generated by the deception analysis service 121. In various embodiments, deception analysis service 121 receives the sensor data captured by interviewee terminal 111 and identifies behavioral cues that correspond to an increased likelihood deception. As part of the behavioral cue identification process, metrics associated with behavioral cues are determined, such as the subject's current response pause time, blink rate, pupil feature, gaze fixation duration, gaze target, heart rate, respiration rate, exhibited facial expressions including micro expressions, and/or temperature fluctuations, among other detected behavioral cues and metrics. In some embodiments, one or more of the metrics are provided at least partially by the sensor equipment and/or by applying deep learning and/or computer vision techniques. For example, visible image data captured using an RGB camera can be fed into a trained deep learning model to predict the subject's heart rate. Similarly, cropped thermal image data of the area surrounding a subject's nostrils can be fed into a trained deep learning model to predict the subject's respiration rate. In some embodiments, computer vision techniques are applied, for example, as another technique for predicting the subject's respiration rate using captured thermal images. In various embodiments, the different determined and predicted metrics associated with the identified behavioral cues are used to determine a deception assessment result, such as a deception assessment score, associated with the subject. A deception assessment result can be provided for each response to a question, for portions of a subject's response to a question, and/or as an overall assessment of the subject over the entire interview.

In some embodiments, deception analysis service 121 stores previously conducted interviews and their results for later retrieval, for example, using an encrypted data store. For example, as a service accessed via a network client, deception analysis service 121 can allow an operator to view past interviews and their corresponding deception analysis results. In some embodiments, the client device used to access the stored interviews is interviewer terminal 101 and/or another properly equipped client device. In some embodiments, the client is a web browser client with the appropriate access permissions to view past interviews.

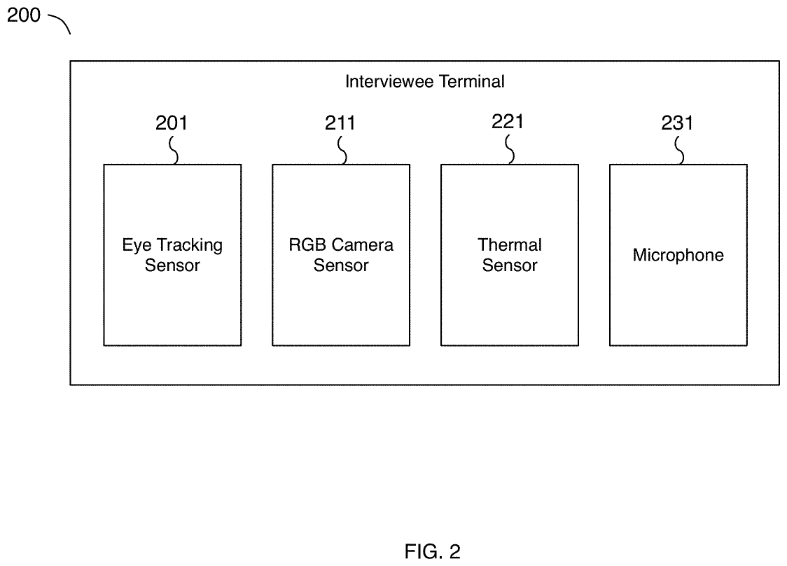

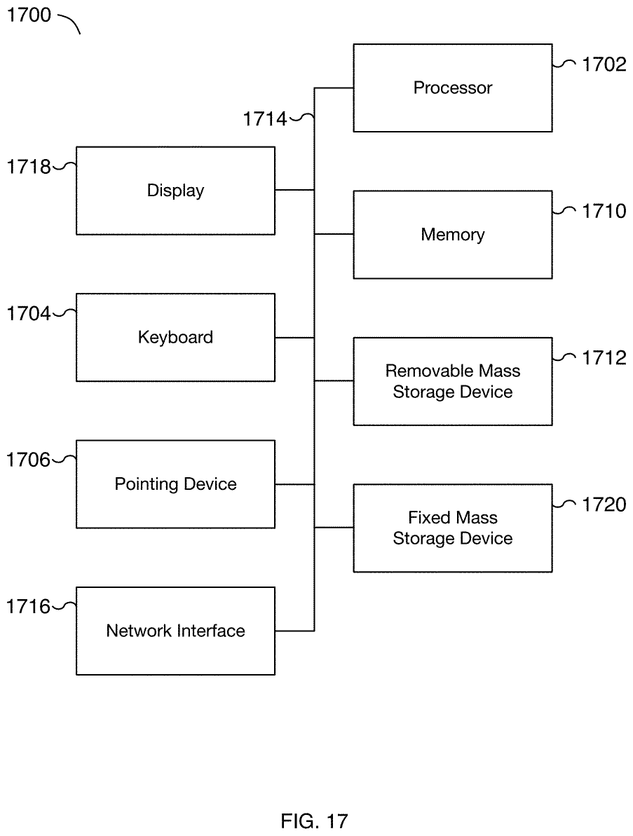

FIG. 2 is a block diagram illustrating sensor components of an embodiment of an interviewee terminal for a multispectral deception analysis system. In the example shown, FIG. 2 displays different non-invasive sensor components for interviewee terminal 200 including eye tracking sensor 201, RGB camera sensor 211, thermal sensor 221, and microphone 231. In some embodiments, fewer or additional sensor components are included. In various embodiments, interviewee terminal 200 is a programmed computer system that includes a display, input and output devices such as a keyboard, mouse, trackpad, and/or touchscreen, and one or more processors and memory components. In some embodiments, interviewee terminal 200 is interviewee terminal 111 of FIG. 1 and is a programmed computer system as described with respect to FIG. 17.

As shown in FIG. 2, interviewee terminal 200 includes four different contactless and non-invasive sensor components. Each of the sensor components is configured to capture sensor data that can be analyzed to determine behavioral cues associated with deception. The sensors are configured to capture their corresponding sensor data without being physically attached to the subject. In various embodiments, the captured sensor data is analyzed by one or more processing components (not shown) of interviewee terminal 200, by an interviewer terminal such as interviewer terminal 101 of FIG. 1, and/or by a deception analysis service such as deception analysis service 121.

In some embodiments, eye tracking sensor 201 is an eye tracking device configured to capture pupil features. For example, data captured using eye tracking sensor 201 can be used to determine pupil changes, including changes in pupil dilations and constrictions, over time. In some embodiments, data captured using eye tracking sensor 201 is used to determine a gaze target and/or a gaze fixation duration. For example, using eye tracking sensor 201, a determination can be made whether the subject is focused on a particular location and/or looking towards a particular target. Additionally, using eye tracking sensor 201, a determination can be made regarding the duration in time that a subject is fixated on the particular target. In various embodiments, other pupil features and related behavioral cues can be detected using sensor data from eye tracking sensor 201. In some embodiments, eye tracking sensor 201 is implemented using an IR sensor and/or an RGB camera. For example, an IR sensor can be utilized as part of an eye tracking sensor to accurately capture eye tracking data for a large range of different eye types including different eye colors.

In some embodiments, RGB camera sensor 211 captures visible light image data. Using the sensor data captured by RGB camera sensor 211, a subject's heart rate and facial expression can be detected. For example, by applying a machine learning model to visible light image data captured using RGB camera sensor 211, the subject's heart rate can be determined. In some embodiments, one or more machine learning models are used to identify facial expressions and combinations of facial expressions exhibited by the subject. The identified facial expressions include micro expressions such as a mouth shrug, a shoulder shrug, a lip press, and a chin raise, among others. In various embodiments, other behavioral cues can be detected using sensor data from RGB camera sensor 211. In some embodiments, RGB camera sensor 211 is used to pinpoint split-second facial movements and identify changes in facial features over time.

In some embodiments, thermal sensor 221 captures thermal image data. In some embodiments, thermal sensor 221 captures infra-red light and is used to record the temperature of the subject. The data can be used to measure minute temperature changes on the surface of the skin to estimate respiration rate and various physiological states. For example, using the sensor data captured by thermal sensor 221, a subject's respiration rate and temperature fluctuations can be detected. In some embodiments, a machine learning model is applied to thermal data captured using thermal sensor 221 to predict the subject's respiration rate. As another example, by analyzing captured thermal data, temperature fluctuations particularly in the subject's face can be detected and analyzed for behavioral cues such as increased blood flow to the nose. In various embodiments, other behavioral cues can be detected using sensor data from thermal sensor 221.

In some embodiments, microphone 231 captures audio data. Using the sensor data captured by microphone 231, a subject's audio response to interview questions can be captured. For example, audio data including the subject's verbal answers as well as other audio behavioral cues can be captured and analyzed to determine the likelihood the subject is being deceptive.

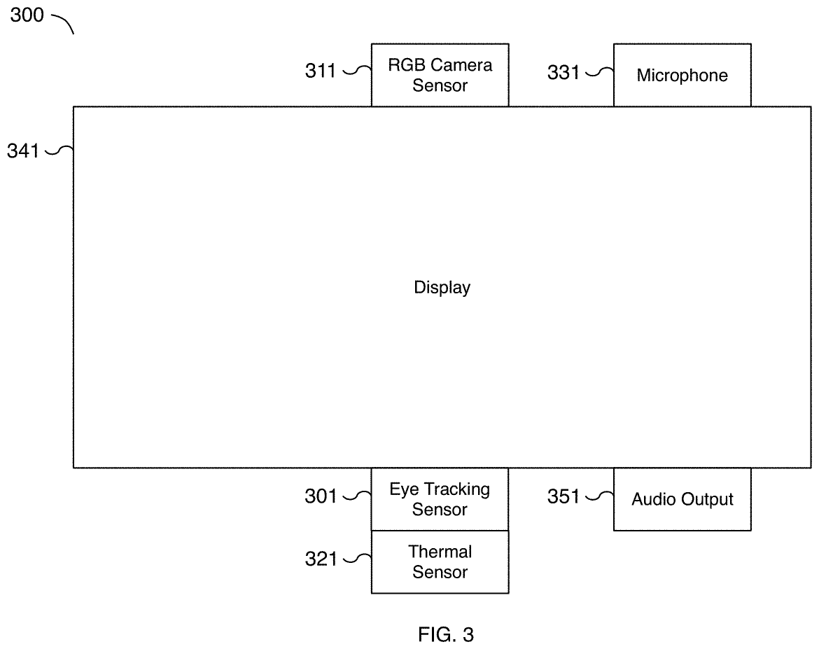

FIG. 3 is a block diagram illustrating the arrangement of different sensor and output components an embodiment of an interviewee terminal for a multispectral deception analysis system. In the example shown, different sensor components and output components of interviewee terminal 300 are shown. The example of FIG. 3 corresponds to the arrangement of the components with a subject facing towards the interviewee terminal 300. In the example shown, interviewee terminal 300 includes eye tracking sensor 301, RGB camera sensor 311, thermal sensor 321, microphone 331, display 341, and audio output 351. Other components of interviewee terminal 300 including other sensor and output components can exist but are not shown. In some embodiments, fewer components including fewer sensors exist. In some embodiments, interviewee terminal 300 is interviewee terminal 111 of FIG. 1 and/or interviewee terminal 200 of FIG. 2. In some embodiments, eye tracking sensor 301, RGB camera sensor 311, thermal sensor 321, and microphone 331 are eye tracking sensor 201, RGB camera sensor 211, thermal sensor 221, and microphone 231, respectively, of FIG. 2.

In the example of FIG. 3, an interview subject faces interviewee terminal 300 and reads the interviewee user interface shown on display 341. Since interviewee terminal 300 is a contactless interviewee terminal, physical contact with the sensors of interviewee terminal 300 can be avoided. For example, the only physical contact that may be required is the use of a conventional input device such as a mouse, touchpad, and/or touchscreen. In various embodiments, display 341 provides a sequence of questions that can include questions that are automatically generated by a deception analysis service and/or provided by an interviewer. In response to each question, the subject provides a verbal response that is captured by microphone 331 and/or another form of a response such as a manual input response. For example, a manual response can be provided by the subject by selecting between multiple answer choices displayed on the interviewee user interface shown on display 341 via a mouse selection and/or touchscreen selection. The subject's other behavioral responses are concurrently captured by the other sensors of interviewee terminal 300 including eye tracking sensor 301, RGB camera sensor 311, and thermal sensor 321.

As shown in FIG. 3, eye tracking sensor 301 can be positioned below or along the bottom of display 341 and RGB camera sensor 311 can be positioned above or along the top of display 341. In some embodiments, RGB camera sensor 311 is placed above display 341 in order to capture the entire face of the interviewee subject for optimal capturing of micro expressions. Thermal sensor 321 is positioned below display 341 and is pointed upwards towards the intended seating position of the interviewee subject. In various embodiments, thermal sensor 321 is positioned below the subject's face and directed at an upward angle to capture the face of the subject. For example, prior to starting an interview, the position of thermal sensor 321 is confirmed and/or adjusted to capture the subject such that thermal sensor 321 is facing upwards towards the subject's face and directed to capture at least the subject's breathing (e.g., nostrils and/or area around the nose). In some embodiments, thermal sensor 321 placement is below display 341 with a slight tilt upwards to be able to clearly look into the nostrils of the interviewee subject in order to determine the respiration rate. In various embodiments, microphone 331 can be positioned along the top (as shown) or bottom of display 341 and audio output 351 can be positioned along the bottom (as shown) or top of display 341.

In some embodiments, multiple instances of the sensor and output components shown in FIG. 3 can exist. For example, microphone 331 and audio output 351 can each exist in pairs to provide stereo audio recording and stereo audio output, respectively. As another example, multiple instances of RGB camera sensor 311 can exist to record different images of the subject, such as at different resolutions, at different frame rates, in different color spaces, and/or from different perspectives.

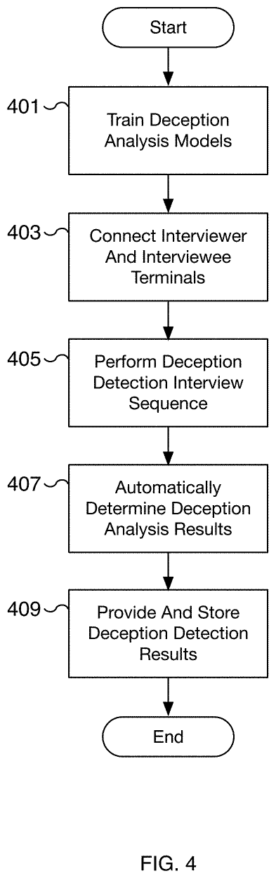

FIG. 4 is a flow chart illustrating an embodiment of a process for assessing deception in a subject. For example, the process of FIG. 4 is performed by a multispectral deception analysis system to detect the likelihood a subject is being deceptive. Using the multispectral deception analysis system, an interview can be initiated, overseen, and/or managed by an interviewer from an interviewer terminal while the interview subject is positioned in front of an interviewee terminal. The interviewee subject's responses are captured by the interviewee terminal and analyzed by a deception analysis service, which provides the results in real time to the interviewer terminal. In some embodiments, the interviewer terminal is interviewer terminal 101 of FIG. 1, the interviewee terminal is interviewee terminal 111 of FIG. 1, interviewee terminal 200 of FIG. 2, and/or interviewee terminal 300 of FIG. 3, and the deception analysis service is deception analysis service 121 of FIG. 1.

At 401, deception analysis models are trained. For example, deep learning models used for predicting deception analysis results are trained. In some embodiments, different models are trained for each set of sensor data and/or deception analysis metric. For example, a deep learning model can be trained for predicting respiration rate using thermal sensor data as input data. As another example, a deep learning model can be trained for predicting facial expressions using RGB camera data as input data. In some embodiments, one or more deep learning models are trained using two or more different sensor data as input.

At 403, interviewer and interviewee terminals are connected. For example, a network connection is established between the interviewer terminal and the interviewee terminal as part of the process for initiating an interview. In various embodiments, the connection is established by an interviewer at the interviewer terminal via a deception analysis service. For example, the interviewer terminal and interviewee terminal can each communicate with one another via the deception analysis service. Both the interviewer terminal and interviewee terminal can function as clients to the deception analysis service. In some alternative embodiments, the interviewer terminal and interviewee terminal do not utilize the deception analysis service as an intermediary. For example, the interviewer terminal can establish a connection with the deception analysis service and then the interviewer terminal guides the interviewee terminal through the different steps of the interview. As another embodiment, the interviewee terminal can establish a connection with the deception analysis service and functions to guide both the interviewer and interviewee terminals through the different steps of the interview.

At 405, a deception detection interview sequence is performed. For example, an interview with a subject positioned at the interviewee terminal is initiated. During the process of the interview, the subject is presented with questions and their responses are captured for analysis. In various embodiments, the interview can be a live interview, an automated interview, or a combination of the two.

At 407, deception analysis results are automatically determined. For example, during the course of the interview, the subject's responses are captured and automatically analyzed. In some embodiments, the sensor data is analyzed by applying the models trained at 401 and/or by applying computer vision techniques. For example, a classifier can map the input variable values to the outcomes of y where σ(y)={lying, inconclusive, truthful} and predict corresponding confidence scores. In some embodiments, a convolutional neural network is utilized that includes image preprocessing layers to account for video temporal dependencies and dense layers and sigmoid layers for binary classification.

In some embodiments, one or more metrics associated with the deception analysis are determined. For example, the metrics determined can correspond to behavioral cues associated with deception. In some embodiments, the analysis results correspond to the likelihood the subject is being deceptive and is determined by the deception analysis service.

At 409, deception analysis results are provided and stored. For example, the results determined at 407 are provided to the interviewer terminal and/or stored for later review. In various embodiments, the results correspond to one or more indicators associated with a likelihood that the subject is being deceptive. For example, an indicator can be presented to the interviewer via a user interface shown on the interviewer terminal. In some embodiments, the provided results include one or more indicators corresponding to behavioral metrics and/or an overall deception assessment metric. In some embodiments, the metrics can be associated with the subject's blink rate, pupil features, heart rate, respiration rate, facial expressions including micro expressions, and/or temperature fluctuations, among other detected behavioral cues. In various embodiments, the stored results can be stored by and later accessed via the deception analysis service.

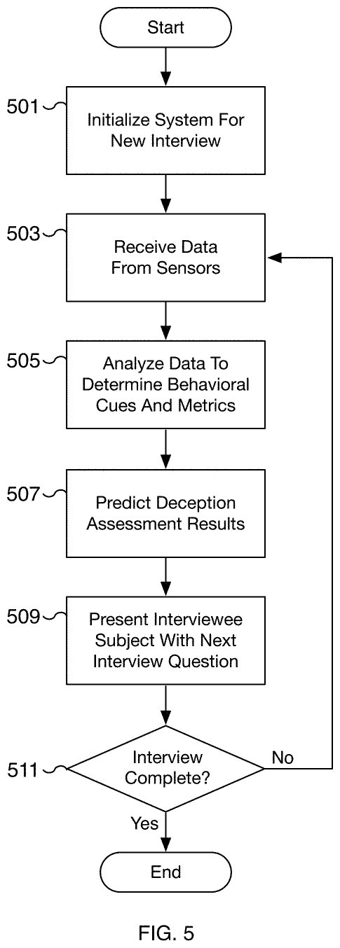

FIG. 5 is a flow chart illustrating an embodiment of a process for performing an interview to assess the likelihood of deception in a subject's responses. For example, the process of FIG. 5 is performed as part of an interview process where the interviewee subject's responses are captured by sensors at an interviewee terminal. In some embodiments, the interviewee terminal is equipped with sensors such as an eye tracking sensor, an RGB camera, a thermal sensor, and/or a microphone to capture different components of the subject's responses. In various embodiments, the interview is initiated by an interviewer at an interviewer terminal and utilizes a deception analysis service to guide the interview and to analyze the likelihood the subject is being deceptive in their responses. In some embodiments, the process of FIG. 5 is performed at 403, 405, and/or 407 of FIG. 4. In some embodiments, the interviewer terminal is interviewer terminal 101 of FIG. 1, the interviewee terminal is interviewee terminal 111 of FIG. 1, interviewee terminal 200 of FIG. 2, and/or interviewee terminal 300 of FIG. 3, and the deception analysis service is deception analysis service 121 of FIG. 1.

At 501, the deception analysis system is initialized for a new interview. For example, the subject is positioned in front of the interviewee terminal and the sensors are configured such that they are correctly positioned to capture the interviewee subject's responses. In various embodiments, the interviewer is positioned in front of the interviewer terminal and confirms that the interview should proceed. For example, the interviewer can input background and/or profile data about the subject, confirm that the interviewee is properly setup for an interview, and/or select the type of interview to perform, such as a live interview or a fully automated interview. In some embodiments, the interviewee inputs their background and/or profile data via the interviewee terminal and can confirm that they are ready for the interview to begin. In various embodiments, network connections are established between interviewer terminal, interviewee terminal, and the deception analysis service such that questions can be presented to the subject and the subject's answers can be captured and analyzed.

At 503, data from sensors is received. For example, data from the different configured sensors of the interviewee terminal is received and captured in preparation for analysis. In some embodiments, eye tracking data including data corresponding to pupil features is received from an eye tracking sensor and visible image data is received from an RGB camera sensor. In some embodiments, thermal image data is received from a thermal sensor and/or audio data is received from a microphone. In various embodiments, the capture of sensor data begins once the interview has started and can start before the first question of the interview has been asked. Along with capturing sensor data, the timing associated with the capture data is tracked as well, for example, to determine a subject's response time for each question. Depending on the configuration of the deception analysis system, the captured sensor data may be transmitted to the deception analysis service for analysis at 505. In some embodiments, the captured sensor data may be transmitted to the interviewer terminal for at least a portion of the analysis and/or is stored locally, at least temporarily, for local processing by the interviewee terminal.

At 505, the sensor data is analyzed to determine behavioral cues and metrics. For example, the captured sensor data is analyzed to identify behavioral cues associated with deception. In some embodiments, the identification of behavioral cues includes determining metrics associated with the behavioral cues. For example, by applying deep learning techniques and/or computer vision techniques, one or more metrics can be determined to identify relevant behavioral cues associated with a likelihood the interviewee subject is being deceptive. Depending on the particular sensor data, different behavioral cues and metrics are determined. For example, using eye tracking data, the fixation duration, gaze target, and/or pupil changes of the subject can be determined. As another example, using an RGB camera, the subject's blink rate can be determined. Additional metrics can include response pause time, response time, respiration rate, heart rate, and the timing and occurrences of facial expressions including micro expressions, among other behavior cues and their associated metrics.

In various embodiments, the sensor data for a response is first identified for relevancy and then further analyzed if potentially relevant. For example, data before the first question and between question responses may be analyzed to determine their relevance and may be discarded if not relevant. In particular embodiments, some preprocessing of the data may be performed as part of the analysis process to determine behavioral cues and associated metrics. Depending on the behavioral cue, different types of preprocessing of the sensor data may be appropriate. In some embodiments, the sensor data is cropped to highlight key features. For example, sensor data around the subject's nose and nostrils can be emphasized to detect respiration rate. As another example, sensor data around the subject's eyes can be emphasized to detect behavioral cues associated with gaze or pupils.

At 507, deception assessment results are predicted. For example, using the behavioral cues and metrics determined at 505, one or more deception assessment results are predicted for the subject's response. In some embodiments, a deception assessment result is predicted using the combination of behavioral cues and associated metrics determined at 505. For example, an elevated respiration rate combined with a pupil feature falling within a certain configured threshold and a blink rate that exceeds a configured threshold value is used to predict a deception assessment value. As another example, a response time that exceeds a configured threshold value along with a gaze fixation that exceeds a configured time length and a combination of detected facial micro expressions is used to predict a deception assessment value. In various embodiments, the predicted value can be a metric such as a percentage value, a rating, a ranking, a Boolean value, or another metric or indicator.

In some embodiments, the deception detection results are determined using prior probabilities. The prior probabilities can be estimated from empirical observations and available datasets. In some embodiments, the results outputted using prior probabilities are subsequently used to update the probabilities using the newly observed data including detected behavioral cues associated with deception. For example, let P(A|B) be the posterior probability of deception (event A) given a new input: deception cue B. Then P(A|B)=(P(A)×P(B|A))/P(B). Accordingly, in some embodiments, the deception detection analysis combines the deception cues and outputs an overall likelihood of deception.

At 509, the interviewee subject is presented with the next interview question. For example, the next interview question is presented to the interviewee subject as part of the interview sequence. In some embodiments, the question is presented via a text prompt on a display of the interviewer terminal. For example, an interviewer user interface displays the next interview question and allows the interviewee subject to respond with an audio response and/or by selecting from one or more choices via an interviewee user interface. In some embodiments, the interviewee subject selects a response using a mouse, trackpad, touchscreen, or another selection device to provide a direct answer response to the interview question. In various embodiments, the interviewee subject is presented with either an automated question, such as an automatically generated question provided by the deception analysis service, or with a question provided by the interviewer. In some embodiments, the interviewer is provided with a selection of questions and selects the current question to present to the interviewee subject.

In various embodiments, the subject is presented with the next interview question only after the subject has completed their previous response. For example, a pause is inserted between questions to allow the subject to reset to a baseline condition. In some embodiments, the pause between questions is based on a time interval, such as a configured time interval. For example, an example time interval to allow a certain pupil feature of the subject to return to a baseline condition is 7 seconds while an example time interval to allow the subject's heart rate to return to a baseline condition is 10 seconds. In some embodiments, the pause between questions is based on sensor data, such as the subject's predicted respiration rate and/or heart rate returning to a baseline metric. In various embodiments, the next interview question is presented to the subject only after the subject's baseline condition is reached.

In some embodiments, the question presented to the subject is part of a guilty knowledge test that also examines the subject's fixation when shown pictures of items that have been smuggled. The delta in fixation between being shown a “guilty” picture similar to what was smuggled vs an item that they did not smuggle is examined. In various embodiments, the images are controlled for luminance effects. The baseline and guilty knowledge test questions are alternated at a 2:1 baseline to guilty knowledge test question ratio across the question set to illicit a greater delta in the subject's cognition. In some embodiments, baseline questions include basic mathematical sums, personal facts, and general knowledge questions. The questions are repeated three times with each question set having a different order. In various embodiments, a pause, such as a 10 second pause, is inserted after the subject answers every question.

At 511, a determination is made whether the interview is complete. For example, a determination is made whether there are any remaining questions to ask the subject and whether the subject has completed their responses to the questions. In the event a determination is made that the interview is complete, processing with respect to the process of FIG. 5 ends. In the event a determination is made that the interview is not complete, processing loops back to 503.

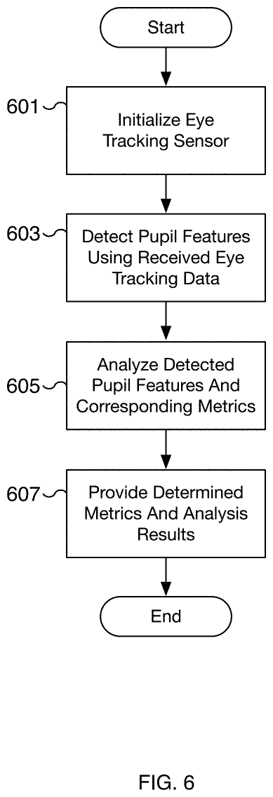

FIG. 6 is a flow chart illustrating an embodiment of a process for analyzing eye tracking sensor data for determining behavioral cues. For example, using the process of FIG. 6, sensor data from an eye tracking sensor of an interviewee terminal is utilized to determine behavioral cues and associated metrics. In some embodiments, the eye tracking sensor is but one of multiple sensors of an interview terminal and the results from analyzing the determined behavioral cues and associated metrics are used as an input for predicting one or more deception assessment results. In some embodiments, the processing and analysis of the sensor data is performed at least in part by an interviewee terminal, interviewer terminal, and/or a deception analysis service and is performed continuously until the interview is complete. For example, the sensor data can be continuously transmitted to a deception analysis service for performing a looping and real-time analysis on the newly captured sensor data until the interview ends. In some embodiments, the process of FIG. 6 is performed at 403, 405, and/or 407 of FIG. 4 and/or at 501, 503, and/or 505 of FIG. 5, and results are used as an input to step 507 of FIG. 5. In some embodiments, the interviewer terminal is interviewer terminal 101 of FIG. 1, the interviewee terminal is interviewee terminal 111 of FIG. 1, interviewee terminal 200 of FIG. 2, and/or interviewee terminal 300 of FIG. 3, and the deception analysis service is deception analysis service 121 of FIG. 1.

At 601, the eye tracking sensor is initialized. For example, at the start of an interview, the eye tracking sensor is initialized for tracking and capturing pupil data. In some embodiments, the initialization includes an initialization sequence that requires the subject to perform a series of configuration steps. The initialization sequence can be used to identify the location of the pupils and to form a baseline for pupil feature extraction. For example, once a change from the baseline pupil is determined, and if the pupil feature falls within a configured threshold, a pupil feature can be tracked to determine when the feature exceeds a configured threshold value. In some embodiments, determining baseline metrics may include performing one or more of the steps of the process of FIG. 6 including steps 603 and/or 605. In some embodiments, the subject is asked to look at certain reference points in order to initialize the system for determining gaze targets. In various embodiments, a subject's baseline metrics can be stored and used for subsequent interviews and can be used to determine when to proceed to the next question in the interview.

At 603, one or more pupil features are detected using the received eye tracking data. For example, sensor data from the initialized eye tracking sensor is captured and used to detect one or more pupil features. In some embodiments, the sensor data is analyzed using computer vision techniques to determine the corresponding metrics for each pupil feature, a duration length for a fixed gaze, and a rate of blinks per second. In some embodiments, the sensor data is used as an input to a deep learning model to predict the corresponding metric. In various embodiments, each determined metric corresponds to a behavioral cue associated with a likelihood the subject is being deceptive.

At 605, the detected pupil features and corresponding metrics are analyzed. For example, detected pupil feature metrics can be compared to configured threshold values. In some embodiments, each detected pupil feature metric is compared with a configured threshold value for that feature. The configured threshold value can correspond to the threshold limit where the detected pupil feature likely contributes to a determination that the subject is being deceptive. For example, a pupil feature threshold can be utilized to determine when the subject's determined pupil feature exceeds a threshold value that indicates there is a likelihood that the subject is being deceptive. Similarly, the existence of a particular gaze target, such as one outside a threshold perimeter defining a focus area, and one held for a particular duration that exceeds a threshold duration length can indicate that there is a likelihood the subject is being deceptive. In various embodiments, the results from analyzing the detected pupil features and corresponding metrics are used to determine whether to utilize certain pupil features in determining a subsequent deception assessment result.

At 607, the determined metrics and analysis results of the sensor data are provided. For example, the analysis results determined at 605 are provided along with the determined pupil feature metrics for subsequent processing. In various embodiments, the amount and type of subsequent processing is determined based on the analysis results. For example, the determined metrics for pupil features can be provided for display in a user interface of the interviewer terminal. As one example, a gaze target that does not indicate deception can be shown on the user interface of the interviewer terminal, however, based on the analysis results, the gaze target metric is not utilized for determining a deception assessment result since the detected gaze did not rise to the level of a deceptive behavioral cue. In various embodiments, one or more metrics of pupil features are provided to the interviewer as indicators shown in the interviewer user interface regardless of whether the corresponding behavioral cues rise to the level of deceptive behavioral cues.

In some embodiments, the pupil features and related metrics that correspond to the identification of a deceptive behavioral cue are also provided for additional subsequent processing in determining deception assessment results. For example, the data provided at 607 can be subsequently combined with one or more other input data determined by analyzing different sensor data and together they can be used as input, such as input features, for determining one or more deception assessment results associated with a subject's response. By combining multiple determined metrics, an analysis can be performed to determine a deception assessment result, such as a deception score associated with the subject's current response. The deception assessment result can be shown on the interviewer user interface as an indicator associated with a likelihood the subject is being deceptive.

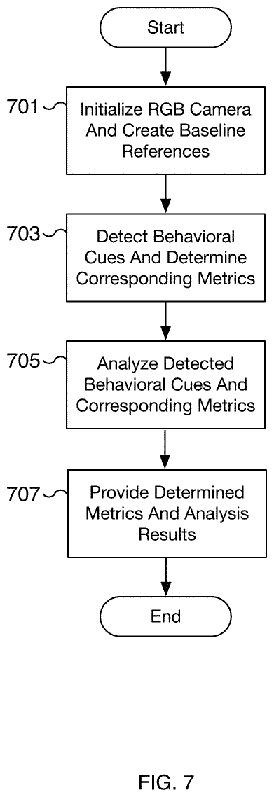

FIG. 7 is a flow chart illustrating an embodiment of a process for analyzing visible image data for determining behavioral cues. For example, using the process of FIG. 7, sensor data from an RGB camera sensor of an interviewee terminal is utilized to determine behavioral cues and associated metrics. In some embodiments, the RGB camera sensor is but one of multiple sensors of an interview terminal and the results from analyzing the determined behavioral cues and associated metrics are used as an input for predicting one or more deception assessment results. In some embodiments, the processing and analysis of the sensor data is performed at least in part by an interviewee terminal, interviewer terminal, and/or a deception analysis service. For example, the visible image sensor data can be transmitted to a deception analysis service for performing the analysis. In some embodiments, the process of FIG. 7 is performed at 403, 405, and/or 407 of FIG. 4 and/or at 501, 503, and/or 505 of FIG. 5, and results are used as an input to step 507 of FIG. 5. In some embodiments, the interviewer terminal is interviewer terminal 101 of FIG. 1, the interviewee terminal is interviewee terminal 111 of FIG. 1, interviewee terminal 200 of FIG. 2, and/or interviewee terminal 300 of FIG. 3, and the deception analysis service is deception analysis service 121 of FIG. 1.

At 701, the RGB camera is initialized and baseline references are created. For example, at the start of an interview, the RGB camera sensor is initialized for capturing visible images of the interviewee subject. Once the RGB camera is initialized, the RGB camera provides a stream and/or sequence of visible image data. In some embodiments, the initialization step includes an initialization sequence that requires the subject to perform a series of configuration steps, such as confirming the subject is within the captured frame of the RGB camera. The initialization sequence can be used to identify the location of key features of the face, such as the eyes, nose, nostrils, and/or mouth, among others and to form one or more baseline measurements, such as a baseline heart rate. In some embodiments, determining baseline metrics may include performing one or more of the steps of the process of FIG. 7 including steps 703 and/or 705. In some embodiments, the subject is asked to look at certain reference points, turn and/or rotate their head, and/or reposition themselves in order to initialize the system. In various embodiments, a subject's baseline metrics can be stored and used for subsequent interviews and can be used to determine when to proceed to the next question in the interview.

In some embodiments, as part of creating baseline references for the subject, features and skin data references are created using received sensor visible image data during the initialization step. For example, references for the color of the subject's skin are created. The skin data references can be used to detect changes in color that corresponding to the subject's heart rate. As another example, facial feature references can be created, such as references of locations for the subject's eyes, nose, nostrils, mouth, lips, shoulders, chin, etc. In some embodiments, a 3D mesh of the subject, such as the 3D model of the subject's face and upper body, is created. Using the created 3D mesh, landmarks of the subject's face can be identified and tracked for movement.

At 703, behavioral cues are detected and corresponding metrics are determined. For example, using the visible image data provided by the RGB camera, behavioral cues associated with a likelihood the subject is being deceptive are detected. The behavioral cues are detected by analyzing the provided visible image data and can include determining corresponding metrics, such as heart rate, blink rate, and/or the existence and/or timing of facial expressions including micro expressions. In some embodiments, the analysis includes preprocessing the image data and applying machine learning and/or computer vision techniques. For example, a training deep learning model can be used to predict the subject's heart rate from changes in the subject's skin color as blood flows under the skin's surface. As another example, facial expressions can be detected by analyzing the movement of facial and body features, such as the subject's lips, chin, eyes, and shoulders, among other features. Metrics associated with an expression can include the time, duration, frequency, and/or number of repeated instances of the expression. In some embodiments, the image data surrounding the subject's eyes is analyzed to determine the subject's blink rate.

In some embodiments, changes are detected by identifying areas on the face using facial landmarks where these appearance changes are the most significant. The data in these regions are accumulated into a three-dimensional data volume. Small-windowed chunks can then be utilized for processing. In some embodiments, signal processing techniques are applied to compute the dominant heart rate within a small time period. In some embodiments, a deep learning network is used to predict the heart beat directly. By utilizing a deep learning approach, the heart rate variability can be minimized.

At 705, the detected behavioral cues and corresponding metrics are analyzed. For example, detected heart rate, blink rate, and/or facial expressions are analyzed to determine whether they meet the threshold level for a deceptive behavioral cue. In some embodiments, the corresponding metrics of the detected behavioral cues are compared to configured threshold values. In some embodiments, a detected heart rate metric is compared with a configured heart rate threshold value. The configured threshold value can correspond to the threshold limit where the subject's heart rate likely contributes to a determination that the subject is being deceptive. Similarly, a detected blink rate metric is compared with a configured blink rate threshold value that can correspond to the threshold limit where the subject's blink rate likely contributes to a determination that the subject is being deceptive. In some embodiments, the facial and body expressions are analyzed to match to known facial/body expressions including micro expressions and combinations of expressions associated with deceptive behavior. In various embodiments, the results from analyzing the detected behavioral cues and corresponding metrics are used to determine whether to utilize the detected behavioral cues and corresponding metrics in determining a subsequent deception assessment result.

At 707, the determined metrics and analysis results of the sensor data are provided. For example, the analysis results determined at 705 are provided along with the determined behavioral cue metrics for subsequent processing. In various embodiments, the amount and type of subsequent processing is determined based on the analysis results. For example, the determined heart rate and/or blink rate metrics can be provided for display in a user interface of the interviewer terminal. As one example, a heart rate that does not indicate deception can be shown on the user interface of the interviewer terminal, however, based on the analysis results, the heart rate metric is not utilized for determining a deception assessment result since the subject's heart rate at the moment did not rise to the level of a deceptive behavioral cue. In various embodiments, one or more behavioral cue metrics are provided to the interviewer as indicators shown in the interviewer user interface regardless of whether the corresponding behavioral cues rise to the level of deceptive behavioral cues.

In some embodiments, the behavioral cues and related metrics that correspond to the identification of a deceptive behavioral cue are also provided for additional subsequent processing in determining deception assessment results. For example, the data provided at 707 can be subsequently combined with one or more other input data determined by analyzing different sensor data and together they can be used as input, such as input features, for determining one or more deception assessment results associated with a subject's response. By combining multiple determined metrics, an analysis can be performed to determine a deception assessment result, such as a deception score associated with the subject's current response. The deception assessment result can be shown on the interviewer user interface as an indicator associated with a likelihood the subject is being deceptive.

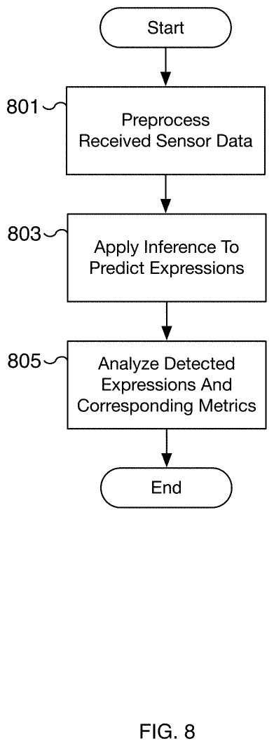

FIG. 8 is a flow chart illustrating an embodiment of a process for analyzing sensor data for determining facial and body expression behavioral cues. For example, using the process of FIG. 8, sensor data such as visible image data from an RGB camera sensor of an interviewee terminal is utilized to determine facial/body expressions and associated metrics. In some embodiments, the process of FIG. 8 is performed at 703 and/or 705 of FIG. 7 by an interviewer terminal, interviewee terminal, and/or deception analysis service. In some embodiments, the interviewer terminal is interviewer terminal 101 of FIG. 1, the interviewee terminal is interviewee terminal 111 of FIG. 1, interviewee terminal 200 of FIG. 2, and/or interviewee terminal 300 of FIG. 3, and the deception analysis service is deception analysis service 121 of FIG. 1.

At 801, the received sensor data is preprocessed. For example, visible image data received from an RGB camera sensor can be optionally preprocessed to extract the portions related to the subject's face and upper body. In some embodiments, the image is preprocessed to remove unnecessary image data, such as the surrounding interview environment or other people who may be in the image such as in the background. In some embodiments, reference points, for example, based on references created during initialization, are used to extract the key portions of the face and body from the sensor data.

At 803, inference is applied to predict expressions. For example, the data preprocessed at 801 is used as input to a machine learning model to predict facial and body expressions. In various embodiments, the input data is visible image data focused on the subject's face and upper body. By applying deep learning techniques using one or more models trained for predicting expressions, a subject's facial and body expression behavioral cues can be detected in the subject's response. The detected expression can include micro expressions. Examples of detected expressions include a lip press, a chin raise, a shoulder shrug, a mouth shrug, and a lip shrug, among others. In some embodiments, only a subset of the detected expressions are associated with deception. In various embodiments, the detected expressions comply with a facial action coding system (FACS).

In some embodiments, deep neural networks are used to predictively label the expressions that the subject makes as facial landmarks. As a facial landmark's position changes, the relative changes for each area of interest are computed. The detection of a visual behavioral cue is triggered once the changes cross a configured threshold. In some embodiments, a deep learning network trained using previously detected visual cues is used to predict complex emotions exhibited by the subject.

In various embodiments, once an expression is detected, metrics associated with the expression are also determined. For example, the time, duration, frequency, and/or number of repeated instances of the expression in the subject's response is determined and associated with each predicted expression. In some embodiments, the detected expression is mapped to the video recording of the subject's response, for example, to allow the video of the subject performing the predicted expression to be annotated with information related to the expression.

At 805, the detected expressions and corresponding metrics are analyzed. For example, each expression is analyzed to determine whether the predicted expression matches an expression associated with a likelihood the subject is being deceptive. In some embodiments, the timing metrics of the detected expressions are compared to determine which expressions overlap or are linked. For example, the combination of certain expressions together can increase the likelihood the subject is being deceptive. In various embodiments, the analysis results along with the detected expressions and corresponding metrics are provided for further processing including for display at the interviewer terminal.

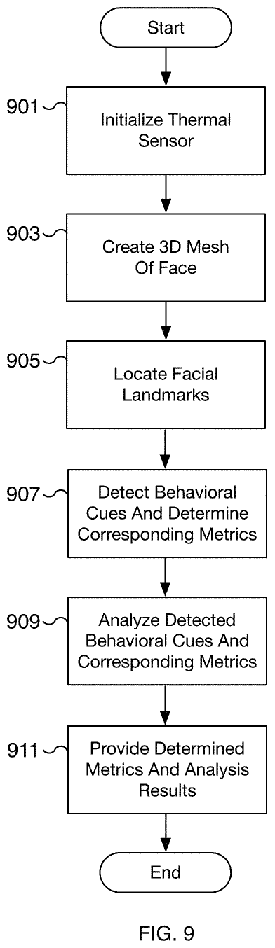

FIG. 9 is a flow chart illustrating an embodiment of a process for analyzing thermal image data for determining behavioral cues. For example, using the process of FIG. 9, sensor data from a thermal sensor of an interviewee terminal is utilized to determine behavioral cues and associated metrics. In various embodiments, the thermal sensor is positioned below the interviewee subject's face and aimed upwards to capture the subject's face and in particular the area that includes the subject's nose and nostrils. In some embodiments, the thermal sensor is positioned to be optimized at least in part for the detection of the subject's respiration rate and/or temperature fluctuations in certain parts of the subject's face. In some embodiments, the thermal sensor is but one of multiple sensors of an interview terminal and the results from analyzing the determined behavioral cues and associated metrics are used as an input for predicting one or more deception assessment results. In some embodiments, the processing and analysis of the sensor data is performed at least in part by an interviewee terminal, interviewer terminal, and/or a deception analysis service. For example, the thermal sensor data can be transmitted to a deception analysis service for performing the analysis. In some embodiments, the process of FIG. 9 is performed at 403, 405, and/or 407 of FIG. 4 and/or at 501, 503, and/or 505 of FIG. 5, and results are used as an input to step 507 of FIG. 5. In some embodiments, the interviewer terminal is interviewer terminal 101 of FIG. 1, the interviewee terminal is interviewee terminal 111 of FIG. 1, interviewee terminal 200 of FIG. 2, and/or interviewee terminal 300 of FIG. 3, and the deception analysis service is deception analysis service 121 of FIG. 1.

At 901, the thermal sensor is initialized. For example, a thermal sensor such as a thermal camera is configured as part of an initialization process. The initialization process can include positioning and confirming the correct positioning of the sensor. For example, in some embodiments, the thermal camera is positioned below the interviewee subject's face and/or aimed upwards to capture the subject's face from an upwards angle. In various embodiments, the sensor is positioned to capture the area of the subject's face that includes the subject's nose and nostrils from an upwards angle. In some embodiments, the subject is asked to look at certain reference points, turn and/or rotate their head, and/or reposition themselves in order to initialize the system. In various embodiments, baseline measurements are taken of the subject such as thermal readings of the subject's face that can include baseline temperatures. The subject's baseline metrics can be stored and used for subsequent interviews and can be used to determine when to proceed to the next question in the interview.

At 903, a 3D mesh of the subject's face is created. For example, a 3D mesh of the subject, such as the 3D model of the subject's face, is created. In some embodiments, the 3D mesh is created as part of a general initialization process and the 3D mesh is utilized for the analysis of sensor data from multiple sensors, such as for analyzing both thermal sensor data and RGB camera image data. In some embodiments, the mesh is created using the thermal sensor data and/or using other sensor data such as image data and/or a depth data. For example, depth data can be captured using a distance sensor such as a lidar sensor and used at least in part to create a 3D model of the subject's face and/or body. In some embodiments, reference coordinates of the 3D mesh are initialized to map between a coordinate system of the created 3D mesh and the coordinate system of the thermal sensor.

At 905, facial landmarks are located. Using the created 3D mesh, landmarks of the subject's face are located and identified. For example, the subject's nose including the tip of the subject's nose can be located and identified. As another example, the subject's nostrils can be located and identified. Similarly, other landmarks such as the cheeks, forehead, lower chin, upper lip, eyes, and temples, among other facial landmarks can be located and identified. By locating and identifying the different facial landmarks, the changes in thermal values associated with the landmarks can be tracked and subsequent analysis of sensor data can be focused on particular areas of interest. In some embodiments, once the landmarks are located and identified, a baseline temperature for each landmark can be identified.

At 907, behavioral cues are detected, and corresponding metrics are determined. For example, by tracking the thermal metrics for the located facial landmarks, behavioral cues such as respiration rate and a “flushed” nose can be detected. In various embodiments, using the received thermal sensor data, thermal metrics such as temperature changes in facial features are tracked. In some embodiments, thermal metrics for specific facial features, such as the nose and the area around the nostrils are tracked. For example, temperature changes in the tip of the nose can correspond to a rush of blood (i.e., a “flushed” nose) and is an indicator of deceptive behavior. As another example, temperature changes surrounding the nostrils and in particular below the nostrils correspond to the subject's breathing as they inhale and exhale. By tracking the thermal metrics of this area, the subject's respiration rate can be determined. For example, the respiration rate of the subject can be detected from the change in temperature of air traveling through the nose. When the subject breathes, the temperature of the air entering the nose is near room temperature, while that of air exiting the nose is close to body temperature. This change in temperature of the air flow inside the nose causes the temperature of the nose to oscillate in sync with respiration rate. After locating the nostrils on the subject's face and collecting thermal data in that region, signal processing techniques can be applied to detect the frequency of temperature changes in the nostrils and provide an accurate estimate of the subject's respiration rate. In various embodiments, the temperature changes and related thermal metrics are tracked to detect behavioral cues and to determine corresponding metrics associated with a likelihood the subject is being deceptive.

At 909, the detected behavioral cues and corresponding metrics are analyzed. For example, a detected “flushed” nose and/or the subject's respiration rate are analyzed to determine whether they meet the threshold level for a deceptive behavioral cue. In some embodiments, the corresponding metrics of the detected behavioral cues are compared to configured threshold values. In some embodiments, a detected respiration rate metric is compared with a configured respiration rate threshold value. The configured threshold value can correspond to the threshold limit where the subject's respiration rate likely contributes to a determination that the subject is being deceptive. Similarly, detected “flushed” nose metrics are compared with configured threshold values that can correspond to the threshold limits where the change in the temperature of the tip of the subject's nose likely contributes to a determination that the subject is being deceptive. The corresponding metrics can relate to the rate of change and/or the temperature at the tip of the nose. In various embodiments, the results from analyzing the detected behavioral cues and corresponding metrics are used to determine whether to utilize the detected behavioral cues and corresponding metrics in determining a subsequent deception assessment result.

At 911, the determined metrics and analysis results of the sensor data are provided. For example, the analysis results determined at 909 are provided along with the determined behavioral cue metrics for subsequent processing. In various embodiments, the amount and type of subsequent processing is determined based on the analysis results. For example, the determined respiration rate and/or “flushed” nose metrics can be provided for display in a user interface of the interviewer terminal. As one example, a respiration rate that does not indicate deception can be shown on the user interface of the interviewer terminal, however, based on the analysis results, the respiration rate metric is not utilized for determining a deception assessment result since the subject's respiration rate at the moment did not rise to the level of a deceptive behavioral cue. In various embodiments, one or more behavioral cue metrics are provided to the interviewer as indicators shown in the interviewer user interface regardless of whether the corresponding behavioral cues rise to the level of deceptive behavioral cues.

In some embodiments, the behavioral cues and related metrics that correspond to the identification of a deceptive behavioral cue are also provided for additional subsequent processing in determining deception assessment results. For example, the data provided at 911 can be subsequently combined with one or more other input data determined by analyzing different sensor data and together they can be used as input, such as input features, for determining one or more deception assessment results associated with a subject's response. By combining multiple determined metrics, an analysis can be performed to determine a deception assessment result, such as a deception score associated with the subject's current response. The deception assessment result can be shown on the interviewer user interface as an indicator associated with a likelihood the subject is being deceptive.

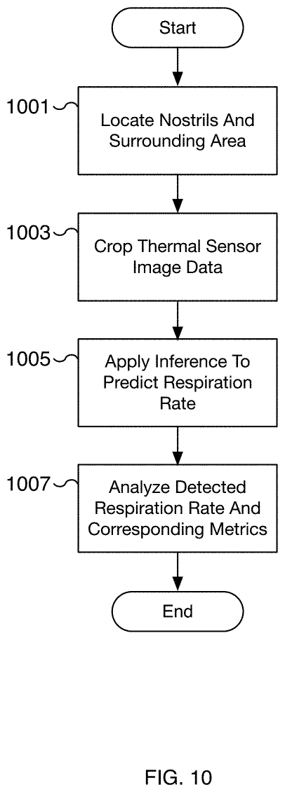

FIG. 10 is a flow chart illustrating an embodiment of a process for analyzing sensor data for determining a subject's respiration rate. For example, using the process of FIG. 10, sensor data such as thermal image data from a thermal sensor of an interviewee terminal is utilized to determine metrics associated with the subject's respiration rate. In some embodiments, the process of FIG. 10 is performed at 905, 907, and/or 909 of FIG. 9 by an interviewer terminal, interviewee terminal, and/or deception analysis service. In some embodiments, the interviewer terminal is interviewer terminal 101 of FIG. 1, the interviewee terminal is interviewee terminal 111 of FIG. 1, interviewee terminal 200 of FIG. 2, and/or interviewee terminal 300 of FIG. 3, and the deception analysis service is deception analysis service 121 of FIG. 1.

At 1001, the subject's nostrils and surrounding area are located. For example, using a 3D mesh of the subject and corresponding thermal sensor data, the areas of the thermal sensor data corresponding to the area surrounding the subject's nostrils are located. In some embodiments, the areas of interest include the areas below the nostrils that correspond to where air enters or leaves the nose.

At 1003, the thermal sensor image data is cropped. For example, the image data is cropped to exclude areas that are not related to the subject's breathing. In various embodiments, the image data is cropped to include only the subject's nostrils and surrounding area as located at 1001. In some embodiments, additional preprocessing is performed on the cropped image data, such as normalizing, quantizing, down sampling, and/or converting the data in preparation for a machine learning inference step.

At 1005, inference is applied to predict the subject's respiration rate. For example, the thermal sensor image data prepared at 1003 is used as input to a machine learning model to predict respiration rate. By applying deep learning techniques using one or more models trained for predicting respiration rate, a subject's respiration rate can be detected in real time as the subject responds to interview questions. The predicted respiration rate can be provided for additional analysis along with one or more respiration rate metrics, such as the change in respiration rate, the duration of the current respiration rate, a maximum detected respiration rate, and/or a baseline or resting respiration rate, among others.

In various embodiments, once a prediction result is determined for the subject's respiration rate, metrics associated with the expression are also determined. For example, the change in respiration rate and the duration of the current respiration rate can be determined. In some embodiments, the detected respiration rate and inhalation/exhalation pattern are used to annotate the video recording of the subject's response. For example, the provided data can allow a recording of the subject to be annotated to show the subject's respiration rate and when the subject is performing different respiration steps such as inhaling and exhaling relative to the subject's interview answers and other behavioral cues.

At 1007, the detected respiration rate and corresponding metrics are analyzed. For example, the subject's detected respiration rate is analyzed to determine whether the predicted respiration rate is associated with a likelihood the subject is being deceptive. In some embodiments, the determination is made by comparing the respiration rate metrics to configured threshold values. In various embodiments, the analysis results along with the detected respiration rate and corresponding metrics are provided for further processing including for display at the interviewer terminal.

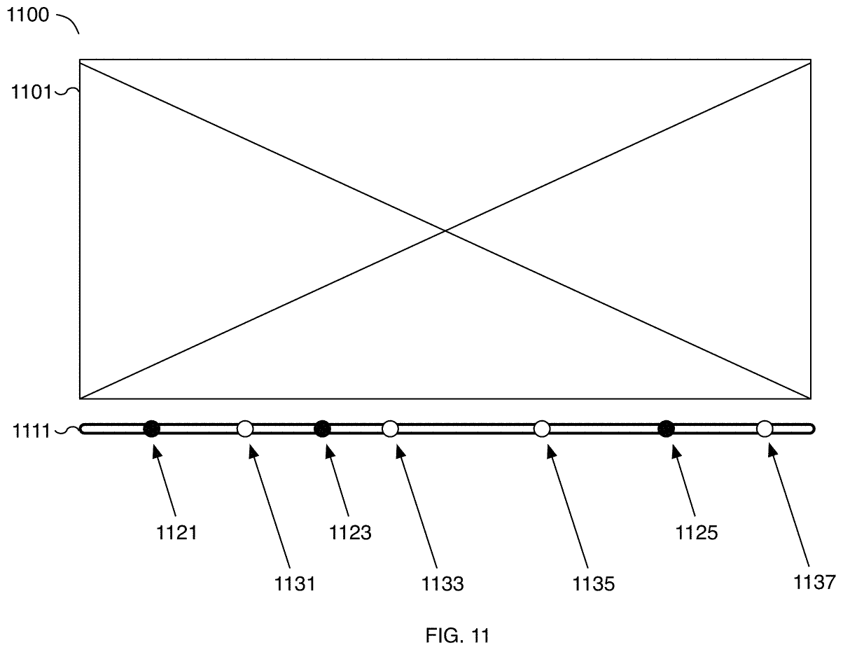

FIG. 11 is a diagram illustrating an embodiment of a playback user interface for viewing key moments of a subject's interview. Using the processes of FIGS. 4-10, a user or operator of an interviewer terminal, such as interviewer terminal 101 of FIG. 1, can review and control the playback of an interview with highlighted key moments. In the example shown, playback user interface 1100 includes video component 1101 and playback highlights component 1111. In various embodiments, playback user interface 1100 is a scroll-based video playback interface that allows an operator to quickly access key moments of an interview. Playback user interface 1100 can be used to view video of an interview in real time and/or post interview.

In some embodiments, video component 1101 is used to display and control the playback of the subject's interview video. For example, a recording of the subject's interview annotated with detected behavioral cues is shown in video component 1101. In some embodiments, additional playback user interface controls such as play, pause, skip, playback speed, and/or replay highlight, among other controls are included (but not shown) in video component 1101. For example, in some embodiments, video component 1101 includes a boomerang user interface control (not shown) to replay a key moment, such as a detected micro expression, in a looped manner and/or in slow motion. In some embodiments, the boomerang user interface control can also allow the viewer to play a deceptive cue in reverse rather than only forward.

In some embodiments, playback highlights component 1111 is a user interface component that highlights the subject's responses. In the example shown, playback highlights component 1111 includes highlighted moments that are bookmarked with highlight moment indicators 1121, 1123, 1125, 1131, 1133, 1135, and 1137. In some embodiments, playback highlights component 1111 allows for instant replay of key moments determined by analyzing the subject's behavior during an interview. For example, an operator can select any of the highlight moment indicators to jump to the associated snippet of the interview video.

In various embodiments, different types of highlight moment indicators are associated with different type of highlights. In the example shown, highlight moment indicators 1121, 1123, and 1125 are differentiated from highlight moment indicators 1131, 1133, 1135, and 1137. In some embodiments, highlight moment indicators 1121, 1123, and 1125 are associated with snippets of the interview determined to be deceptive. The associated snippets of the video associated with highlight moment indicators 1121, 1123, and 1125 can be annotated with the detected behavioral cues that are determined to be associated with deceptive behavior. In some embodiments, highlight moment indicators 1131, 1133, 1135, and 1137 are associated with responses by the subject that were not determined to be deceptive and/or detected behavioral cues that were determined to be not deceptive. In some embodiments, the highlight moment indicators can include additional detail (not shown), such as a preview of the associated snippet, a timestamp, and/or a description of the detected behavior cue if appropriate. In various embodiments, the highlight moment indicators allow the operator to quickly access a key moment of the subject's interview.

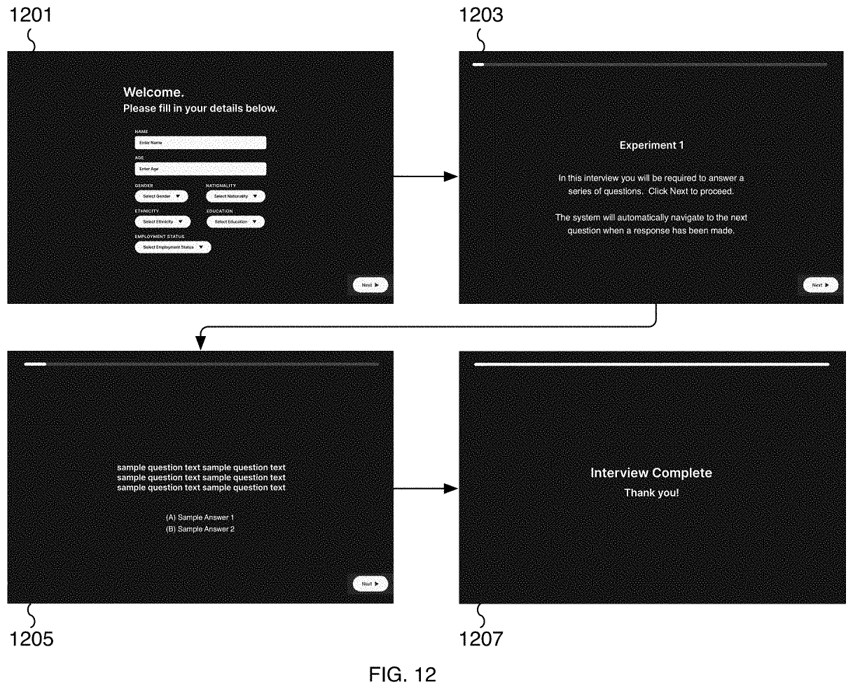

FIG. 12 is a diagram illustrating an embodiment of an interviewee user interface. In various embodiments, an interviewee subject is presented with a user interface such as the user interface of FIG. 12 during an interview. In the example shown, the interviewee user interface includes user interface screens 1201, 1203, 1205, and 1207. The arrows between the different user interface screens indicate a normal progression taken during an interview process. In various embodiments, the interviewee user interface is displayed on an interviewee terminal such as interviewee terminal 111 of FIG. 1, interviewee terminal 200 of FIG. 2, and/or interviewee terminal 300 of FIG. 3. Data for the user interface can be provided and input received from the user interface can be received by an interviewer terminal and/or a deception analysis service. In some embodiments, the interviewer terminal is interviewer terminal 101 of FIG. 1 and the deception analysis service is deception analysis service 121 of FIG. 1. In various embodiments, the interviewee user interface of FIG. 12 is generated and/or utilized during the processes of FIGS. 4-10.

In the example shown, user interface screens 1201, 1203, 1205, and 1207 depict different example user interface screens shown to an interviewee subject. User interface screen 1201 is provided to allow the subject to enter the background details. The received details can be stored by the deception analysis service for later retrieval and/or modification. User interface screen 1203 is an example screen displaying instructions for the subject. User interface screen 1205 is an example screen displaying an interview question. In some embodiments, the question is an open-ended question that allows for a free-formed response. In some embodiments, the question includes a discrete number of responses (e.g., yes or no options or multiple-choice options) and the subject selects from the allowable responses either by using a manual input device such as a mouse, trackpad, and/or touchscreen, with an audible response, and/or with another response such as a head nod. User interface screen 1207 is an example complete screen that is presented to the subject when the interview is complete. In some embodiments, the complete screen may include follow up instructions. As shown in FIG. 12, user interface screens 1203, 1205, and 1207 each include a progress bar on the top of their respective screens that provides the subject with a visual representation of their progress for the interview.

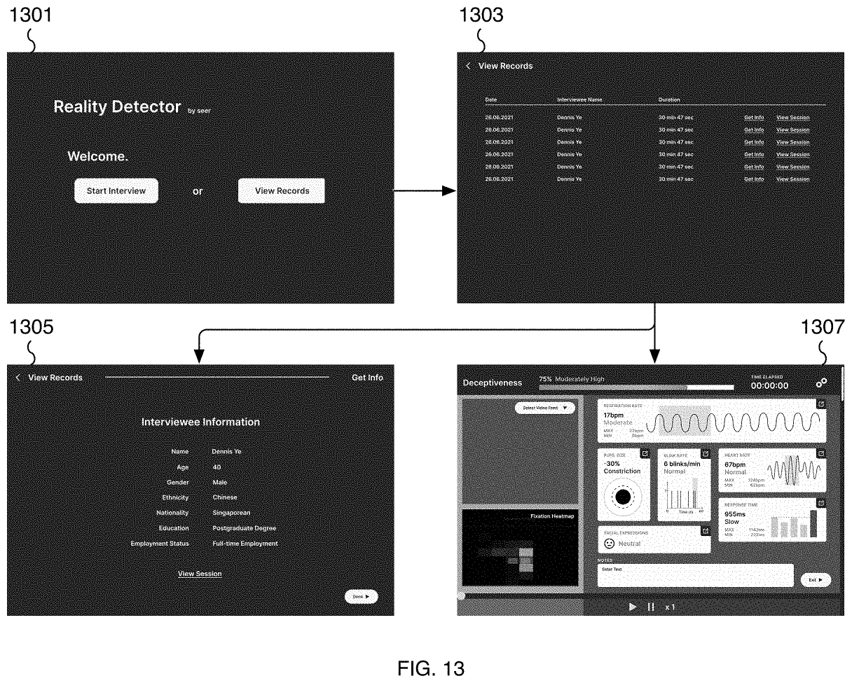

FIG. 13 is a diagram illustrating an embodiment of an interviewer user interface for viewing records. In various embodiments, an interviewer is presented with a user interface such as the user interface of FIG. 13 to review past interviews and the deception analysis performed on those interviews. In the example shown, the interviewer user interface includes user interface screens 1301, 1303, 1305, and 1307. The arrows between the different user interface screens indicate normal progressions taken when retrieving an interview record. In various embodiments, the interviewer user interface is displayed on an interviewer terminal. Data for the user interface can be provided by the interviewer terminal and/or a deception analysis service. For example, the past interviews can be stored on a cloud storage and accessed from the interviewer terminal via the deception analysis service. In some embodiments, the interviewer terminal is interviewer terminal 101 of FIG. 1 and the deception analysis service is deception analysis service 121 of FIG. 1. In various embodiments, the interviewer user interface of FIG. 13 is a user interface for presenting and interacting with interview data and deception analysis results generated using the processes of FIGS. 4-10.

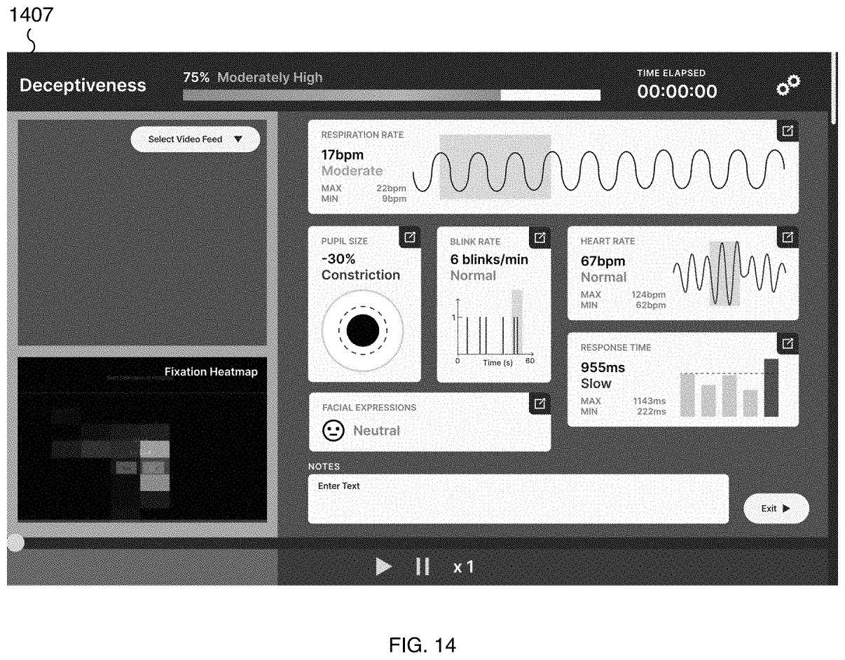

In the example shown, user interface screens 1301, 1303, 1305, and 1307 depict different example user interface screens shown to an interviewee subject for accessing past interviews and the deception analysis performed on those interviews. User interface screen 1301 is a menu interface that allows an operator to select between starting a new interview and viewing a previously saved record. By selecting the “View Records” option, the operator is presented with user interface screen 1303. User interface screen 1303 displays a list of past interviews that the operator has access to. For each accessible past interview, a date, an interviewee name, a duration, a get information action, and a view session action are shown. Selecting the get information action (labeled as “Get Info”) presents the operator with user interface screen 1305 and selecting the view session action (labeled as “View Session”) presents the operator with user interface screen 1307. User interface screen 1305 displays interviewee information for the corresponding interview and user interface screen 1307 displays the corresponding interview as an interview session. In various embodiments, user interface screen 1307 is an embodiment of an interactive view session user interface screen and depicts different behavioral cues and corresponding metrics of the interviewee subject along with one or more selectable video feeds of the subject's interview. In various embodiments, a detailed view of user interface screen 1307 is shown in FIG. 14.

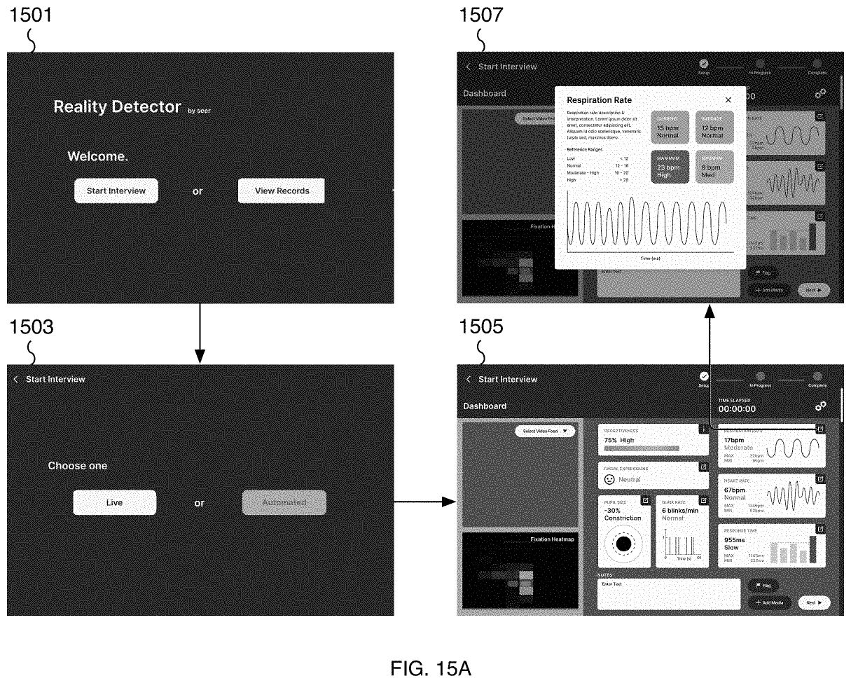

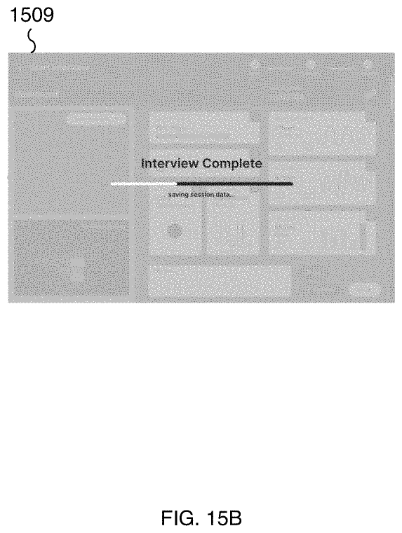

FIG. 14 is a diagram illustrating an embodiment of an interactive view session user interface screen for viewing an interview. In various embodiments, an interviewer is presented with a user interface such as the user interface of FIG. 14 to view past and current interviews and the deception analysis performed for the interviews. For example, when viewing past interviews, interactive view session user interface screen 1407 is a detailed view of an embodiment of user interface screen 1307 of FIG. 13 and when viewing current interviews, interactive view session user interface screen 1407 is a detailed view of an embodiment of user interface screen 1505 of FIG. 15A. In some embodiments, an interactive view session user interface screen for viewing past interviews and current interviews may differ slightly but include many of the same core user interface components. In some embodiments, the data for the user interface is provided by an interviewer terminal and/or a deception analysis service. In some embodiments, the interviewer terminal is interviewer terminal 101 of FIG. 1 and the deception analysis service is deception analysis service 121 of FIG. 1. In various embodiments, the interviewer user interface of FIG. 14 is a user interface for presenting and interacting with interview data and deception analysis results generated using the processes of FIGS. 4-10.

As shown in the example of FIG. 14, interactive view session user interface screen 1407 includes multiple user interface components for viewing an interview and the corresponding deception analysis performed on the subject in the interview. For example, interactive view session user interface screen 1407 includes a user interface component to select between different video feeds of the interview, such as between one or more visible image video feeds and a thermal video feed. As another example, interactive view session user interface screen 1407 includes a fixation heatmap component that highlights where the subject's eyes are focused over time and different user interface components that display detailed metrics associated with the subject's behavioral cues such as respiration rate, pupil feature, blink rate, facial expressions, heart rate, and response time. In some embodiments, the fixation heatmap component displays more grey/red colors as a subject's point of focus directed at particular areas of the interviewer user interface increases. Interactive view session user interface screen 1407 further includes a user interface component to view and enter notes and a user interface component that includes playback controls. For example, using the playback controls, an operator can change the speed of playback and/or replay the subject's earlier responses. When viewing past interviews, the operator can also skip forward in time to later responses. In some embodiments, screen 1407 includes a deceptiveness assessment result indicator such as the deceptiveness score shown along the top of the screen. In the example shown, the deceptiveness metric has the value 75% and is described as “Moderately High.” Interactive view session user interface screen 1407 also includes additional functionality such as the time elapsed user interface component and a configuration user interface component in the upper-right hand of the screen to adjust configuration settings.

FIGS. 15A and 15B are diagrams illustrating an embodiment of an interviewer user interface for performing an interview. In various embodiments, an interviewer is presented with a user interface such as the user interfaces of FIGS. 15A and 15B to initiate and manage an interview and to view the deception analysis performed for the initiated interview. In the example shown, the interviewer user interface includes user interface screens 1501, 1503, 1505, 1507, and 1509. The arrows between the different user interface screens indicate normal progressions taken when retrieving an interview record. An interview is initiated and started from user interface screen 1501 and when the interview completes, user interface screen 1509 is shown. User interface screen 1509 can be reached from user interface screens 1505 or 1507. In various embodiments, the interviewer user interface is displayed on an interviewer terminal. Data for the user interface can be provided by the interviewer terminal and/or a deception analysis service. In some embodiments, the interviewer terminal is interviewer terminal 101 of FIG. 1 and the deception analysis service is deception analysis service 121 of FIG. 1. In various embodiments, the interviewer user interface of FIGS. 15A and 15B is a user interface for presenting and interacting with interview data and deception analysis results generated using the processes of FIGS. 4-10.

In the example shown, user interface screens 1501, 1503, 1505, 1507, and 1509 depict different example user interface screens shown to an interviewer for starting a new interview and then viewing the interview deception analysis results. User interface screen 1501 is a menu interface that allows an operator to select between starting a new interview and viewing a previously saved record. In various embodiments, user interface screen 1501 is user interface screen 1301 of FIG. 13. By selecting the “Start Interview” option, the operator is presented with user interface screen 1503. User interface screen 1503 allows the operator to start a live interview or an automated interview.

In the example of FIGS. 15A and 15B, a live interview is selected, and the operator is presented with user interface screen 1505. User interface screen 1505 is an interactive view session user interface screen. In various embodiments, user interface screen 1505 is an embodiment of user interface screen 1407 of FIG. 14 for viewing live interviews and its functionality is described in further detail with respect to FIG. 14. As shown in FIG. 15A, user interface screen 1505 includes a progress bar that indicates the current stage of the interview among the potential stages that include setup, in progress, and complete. The “Start Interview” label indicates that user interface screen 1505 is invoked from user interface screen 1503 by selecting the “Start Interview” option. Since the interview has not started, user interface screen 1505 does not show a deceptiveness assessment result indicator but does show the currently detected behavioral cues and corresponding metrics for the interviewee subject.

As shown in user interface screen 1505, many of the user interface components can be expanded by selecting an expand icon in the upper-right corner of a corresponding user interface component. For example, when expanding the respiration rate user interface component, user interface screen 1507 is displayed to the operator. User interface screen 1507 is one example of a user interface screen for viewing detailed metrics associated with the subject. In the example shown, user interface screen 1507 displays detailed information and metrics associated with the subject's respiration rate including current rate, average rate, maximum rate, minimum rate, reference ranges, and the rate graphed over time. In some embodiments, the description and interpretation of the detected metrics is also displayed for the operator.

In various embodiments, when the interview completes, the operator is presented with user interface screen 1509 of FIG. 15B. User interface screen 1509 is an example complete screen and includes a progress user interface component. The progress user interface component provides the operator with information relating to the save progress of the interview session. In various embodiments, the interview session and deception analysis results are saved to an online data store such as a cloud data store via a deception analysis service.

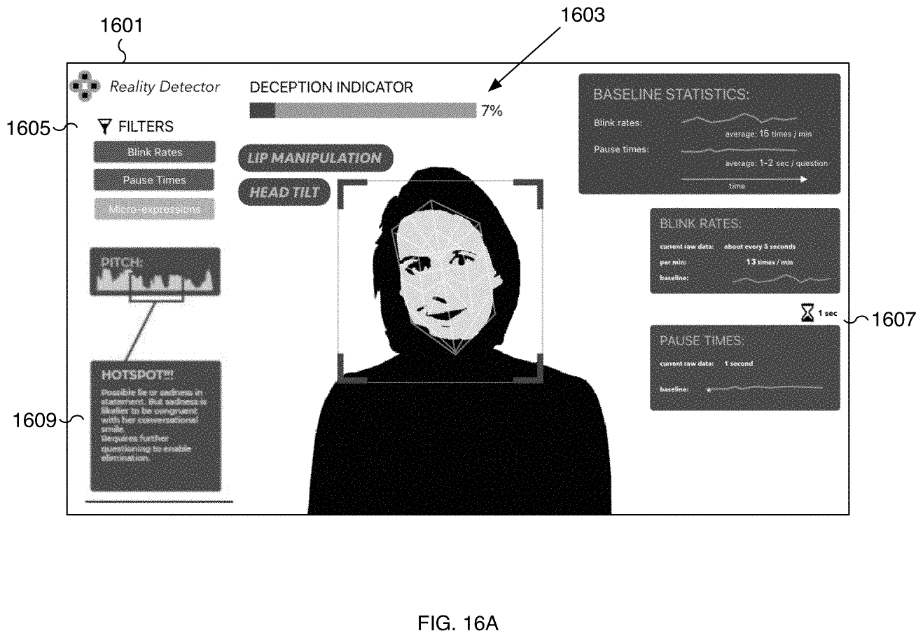

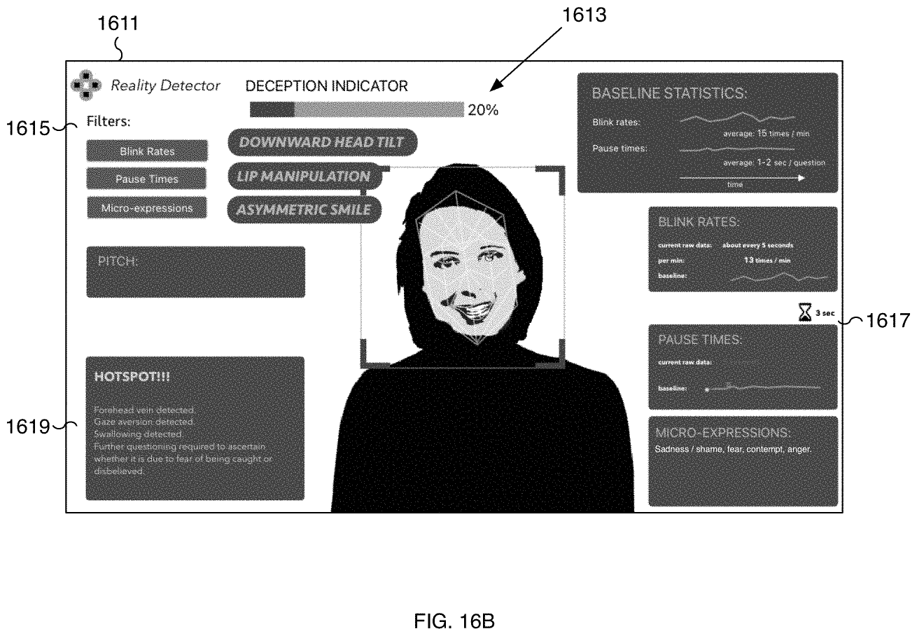

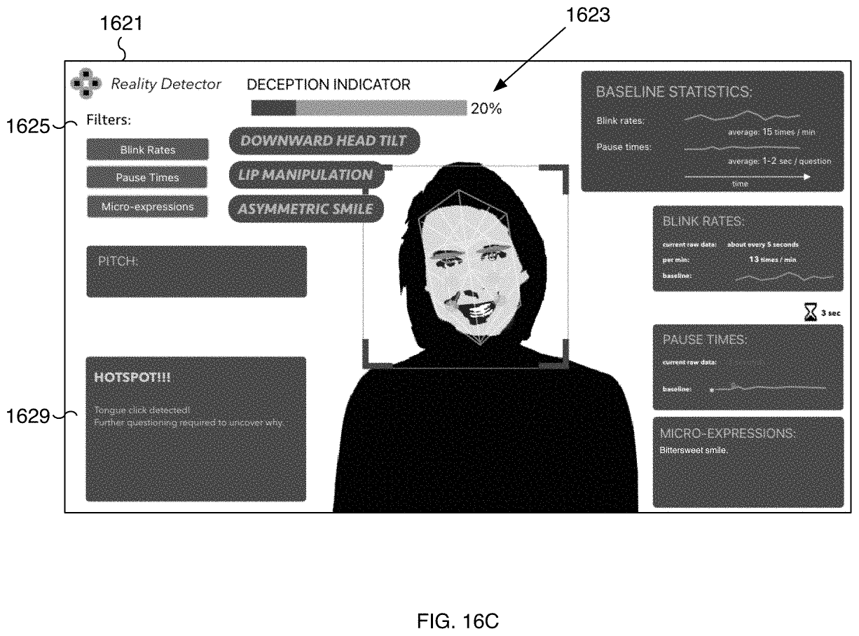

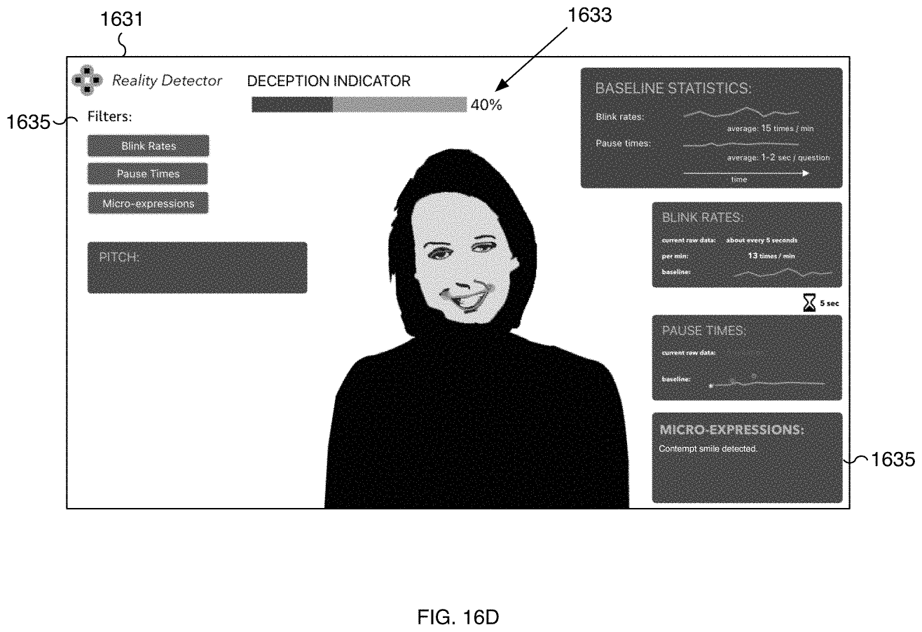

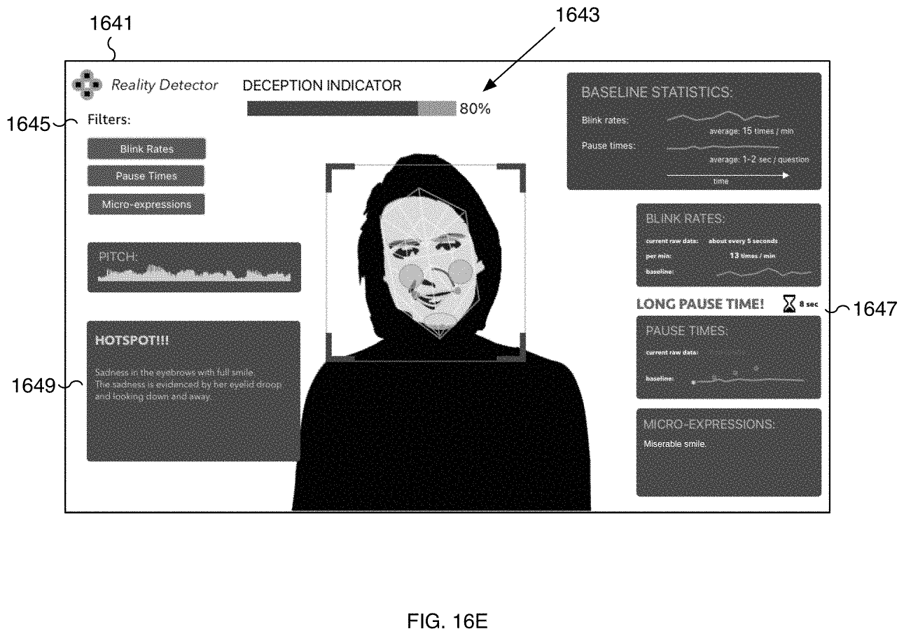

FIGS. 16A-16E are diagrams illustrating an embodiment of an interactive view session user interface screen for viewing an interview. In various embodiments, an interviewer is presented with a user interface such as the user interface of FIGS. 16A-16E to view past and current interviews and the deception analysis performed for the interviews. In some embodiments, an interactive view session user interface screen for viewing past interviews and current interviews may differ slightly but include many of the same core user interface components. In some embodiments, the data for the user interface is provided by an interviewer terminal and/or a deception analysis service. In some embodiments, the interviewer terminal is interviewer terminal 101 of FIG. 1 and the deception analysis service is deception analysis service 121 of FIG. 1. In various embodiments, the interviewer user interface of FIGS. 16A-16E is a user interface for presenting and interacting with interview data and deception analysis results generated using the processes of FIGS. 4-10.

As shown in FIGS. 16A-16E, the example user interface includes user interface screens 1601, 1611, 1621, 1631, and 1641. In various embodiments, the different user interface screens of FIGS. 16A-16E correspond to an interactive view session user interface at different moments of an interview and are shown in temporal order as the interview progresses. In each of user interface screens 1601, 1611, 1621, 1631, and 1641, a deception indicator is shown with a corresponding deception assessment result. Similarly, each of user interface screens 1601, 1611, 1621, 1631, and 1641 includes a filters selection user interface component. For example, user interface screens 1601, 1611, 1621, 1631, and 1641 include deception indicators 1603, 1613, 1623, 1633, and 1643, respectively, and filters selection user interface components 1605, 1615, 1625, 1635, and 1645, respectively.

In the example user interface screens 1601, 1611, 1621, 1631, and 1641, a subset of the user interface components is labeled to help in describing their respective features. Specifically, in a subset of user interface screens, a pause time user interface component, a hotspot user interface component, and/or a micro-expressions user interface component is labeled. For example, user interface screens 1601, 1611, and 1641 include labeled pause time user interface components 1607, 1617, and 1647, respectively, user interface screens 1601, 1611, 1621, and 1641 include labeled hotspot user interface components 1609, 1619, 1629, and 1649, respectively, and user interface screen 1631 includes labeled micro-expressions user interface component 1635. Other components in user interface screens 1601, 1611, 1621, 1631, and 1641 are shown but are not labeled, such as a pitch user interface component, a baseline user interface component, a blink rate user interface component, and the annotate video of the subject.

In various embodiments, user interface screens 1601, 1611, 1621, 1631, and 1641 include deception indicators 1603, 1613, 1623, 1633, and 1643, respectively. Each of deception indicators 1603, 1613, 1623, 1633, and 1643 display the current assessment of the subject's likelihood of deception based on deception detection results. The indicators each include a percentage metric, such as 7% for deception indicator 1603, 20% for deception indicators 1613 and 1623, 40% for deception indicator 1633, and 80% for deception indicator 1643. As the interview progresses, additional behavioral cues associated with a likelihood the subject is being deceptive are detected and the deception metric associated with the deception indicator increases. User interface screens 1601, 1611, 1621, 1631, and 1641 also include filters selection user interface components 1605, 1615, 1625, 1635, and 1645, respectively. Using filters selection user interface components 1605, 1615, 1625, 1635, and 1645, an operator can activate or deactivate the detection and/or display of different types of behavioral cues and also quickly inspect which filters are enabled or disabled. Filters selection user interface components 1605, 1615, 1625, 1635, and 1645 all have a blink rate and pause time filters activated, which corresponds to displaying a blink rate user interface component and a pause time user interface component on their associated view session screens. Similarly, filters selection user interface components 1615, 1625, 1635, and 1645 also enable a micro-expressions filter which enables the display of a micro-expressions user interface component on their associated view session screens.

As shown in the example, user interface screens 1601, 1611, 1621, 1631, and 1641 each display a baseline user interface component. The baseline user interface components show the baseline metrics for the subject. In some embodiments, the baseline metrics are used at least in part to determine when a subject is presented with the next interview question. For example, the deception analysis system waits until a subject's behavioral metrics return to a baseline condition before presenting the next question. In some embodiments, the return to a baseline condition is approximated by a time delay. In the examples shown, the baseline user interface components include metrics for blink rate and pause time metrics but other metrics can be included as well such as metrics for heart rate and respiration rate.

In various embodiments, a pause time user interface component displays the pause time associated with a subject's response. For example, pause time user interface component 1607 of user interface screen 1601 shows 1 second of pause time. In some embodiments, a pause time user interface component includes an icon such as an hourglass icon to indicate the current measured pause time. In some embodiments, the timer measures the subject's response pause time before answering an interview question and resets when the subject answers a question. The timer can be a time that counts down or a timer that counts up. For example, a default countdown time can be configured, for example, with a threshold value to trigger an alert when the countdown is exceeded. In some embodiments, the timer is a count up timer that increases as long as the subject pauses before providing a response. As shown with pause time user interface component 1647 of user interface screen 1641, the timer has reached 8 seconds of pause time and has triggered a long pause time alert. In many scenarios, a detected long pause time is associated with a higher likelihood that the subject is being deceptive. In various embodiments, data and metrics associated with each captured pause time are recorded and shown, for example, as a graph. For example, pause time user interface component 1617 of user interface screen 1611 includes a graph with two previously recorded data points (the first is a baseline data point) and user interface component 1647 of user interface screen 1641 includes a graph with four previously recorded data points.

In some embodiments, a hotspot user interface component is displayed on the view session screen. The hotspot user interface component can call out detected behavioral cues and can provide the operator with additional analysis and potentially actionable steps to take. For example, user interface screens 1601, 1611, 1621, and 1641 include labeled hotspot user interface components 1609, 1619, 1629, and 1649, respectively. Hotspot user interface component 1609 of user interface screen 1601 is an example hotspot user interface component that references another user interface component. The description provided in hotspot user interface component 1609 brings attention to detected pitch metrics that correspond to a likelihood that the subject is being deceptive. As another example, hotspot user interface component 1619 of user interface screen 1611 informs the operator that multiple behavioral cues are detected including a forehead vein, a gaze aversion, and swallowing. Similarly, hotspot user interface component 1629 of user interface screen 1621 informs the operator that a tongue click was detected and hotspot user interface component 1649 of user interface screen 1641 informs the operator that expressions associated with sadness, such as a smile with an eyelid droop while gazing down and away, were detected.

In some embodiments, the facial and body features of the subject in the interview video are annotated with the detected facial and body expressions and/or movements. The overlayed annotations allow the operator to observe the detected expressions. For example, user interface screen 1631 includes a video of the interviewee subject with an annotated smile but no corresponding eye movement. The combination indicates that the subject's smile is likely not genuine. The combination of detected expressions along with the increase in pause time results in an increase of the deception indicator metric to 40% (from 20% as shown in user interface screen 1621). As another example, user interface 1641 includes a video of the subject with an annotated smile and eyebrows that indicate sadness. The combination of detected behavioral cues results in a further increase of the deception indicator metric to 80% (from 40% as shown in user interface screen 1631). The corresponding predicted emotions associated with the detected micro-expressions may be shown in a micro-expressions user interface component such as with user interface screen 1641.

In some embodiments, the interview video of the subject is further annotated with a 3D mesh of the subject. The overlayed 3D mesh of the subject's face allows the operator to visualize asymmetry in the subject's responses. For example, user interface screen 1611 shows the subject with a downward head tilt and a lip manipulation cue that results in an asymmetric smile. In various embodiments, the detection of asymmetric elements, such as the asymmetric smile, indicates a likelihood of contempt. The corresponding predicted emotions associated with the detected asymmetry may be shown in a micro-expressions user interface component such as with user interface screen 1611.

FIG. 17 is a functional diagram illustrating a programmed computer system for performing deception analysis of an interviewee subject. As will be apparent, other computer system architectures and configurations can be utilized for performing deception analysis. Examples of computer system 1700 include interviewer terminal 101, interviewee terminal 111, and one or more computers of deception analysis service 121 of FIG. 1. Computer system 1700, which includes various subsystems as described below, includes at least one microprocessor subsystem (also referred to as a processor or a central processing unit (CPU)) 1702. For example, processor 1702 can be implemented by a single-chip processor or by multiple processors. In some embodiments, processor 1702 is a general purpose digital processor that controls the operation of the computer system 1700. Using instructions retrieved from memory 1710, the processor 1702 controls the reception and manipulation of input data, and the output and display of data on output devices (e.g., display 1718). In various embodiments, one or more instances of computer system 1700 can be used to implement at least portions of the processes of FIGS. 4-10.

Processor 1702 is coupled bi-directionally with memory 1710, which can include a first primary storage, typically a random access memory (RAM), and a second primary storage area, typically a read-only memory (ROM). As is well known in the art, primary storage can be used as a general storage area and as scratch-pad memory, and can also be used to store input data and processed data. Primary storage can also store programming instructions and data, in the form of data objects and text objects, in addition to other data and instructions for processes operating on processor 1702. Also as is well known in the art, primary storage typically includes basic operating instructions, program code, data and objects used by the processor 1702 to perform its functions (e.g., programmed instructions). For example, memory 1710 can include any suitable computer-readable storage media, described below, depending on whether, for example, data access needs to be bi-directional or unidirectional. For example, processor 1702 can also directly and very rapidly retrieve and store frequently needed data in a cache memory (not shown).

A removable mass storage device 1712 provides additional data storage capacity for the computer system 1700, and is coupled either bi-directionally (read/write) or unidirectionally (read only) to processor 1702. For example, storage 1712 can also include computer-readable media such as magnetic tape, flash memory, PC-CARDS, portable mass storage devices, holographic storage devices, and other storage devices. A fixed mass storage 1720 can also, for example, provide additional data storage capacity. The most common example of mass storage 1720 is a hard disk drive. Mass storages 1712, 1720 generally store additional programming instructions, data, and the like that typically are not in active use by the processor 1702. It will be appreciated that the information retained within mass storages 1712 and 1720 can be incorporated, if needed, in standard fashion as part of memory 1710 (e.g., RAM) as virtual memory.

In addition to providing processor 1702 access to storage subsystems, bus 1714 can also be used to provide access to other subsystems and devices. As shown, these can include a display monitor 1718, a network interface 1716, a keyboard 1704, and a pointing device 1706, as well as an auxiliary input/output device interface, a sound card, speakers, and other subsystems as needed. For example, the pointing device 1706 can be a mouse, stylus, track ball, or tablet, and is useful for interacting with a graphical user interface.

The network interface 1716 allows processor 1702 to be coupled to another computer, computer network, or telecommunications network using a network connection as shown. For example, through the network interface 1716, the processor 1702 can receive information (e.g., data objects or program instructions) from another network or output information to another network in the course of performing method/process steps. Information, often represented as a sequence of instructions to be executed on a processor, can be received from and outputted to another network. An interface card or similar device and appropriate software implemented by (e.g., executed/performed on) processor 1702 can be used to connect the computer system 1700 to an external network and transfer data according to standard protocols. For example, various process embodiments disclosed herein can be executed on processor 1702, or can be performed across a network such as the Internet, intranet networks, or local area networks, in conjunction with a remote processor that shares a portion of the processing. Additional mass storage devices (not shown) can also be connected to processor 1702 through network interface 1716.

An auxiliary I/O device interface (not shown) can be used in conjunction with computer system 1700. The auxiliary I/O device interface can include general and customized interfaces that allow the processor 1702 to send and, more typically, receive data from other devices such as microphones, touch-sensitive displays, transducer card readers, tape readers, voice or handwriting recognizers, biometrics readers, cameras, portable mass storage devices, and other computers.

In addition, various embodiments disclosed herein further relate to computer storage products with a computer readable medium that includes program code for performing various computer-implemented operations. The computer-readable medium is any data storage device that can store data which can thereafter be read by a computer system. Examples of computer-readable media include, but are not limited to, all the media mentioned above: magnetic media such as hard disks, floppy disks, and magnetic tape; optical media such as CD-ROM disks; magneto-optical media such as optical disks; and specially configured hardware devices such as application-specific integrated circuits (ASICs), programmable logic devices (PLDs), and ROM and RAM devices. Examples of program code include both machine code, as produced, for example, by a compiler, or files containing higher level code (e.g., script) that can be executed using an interpreter.

The computer system shown in FIG. 17 is but an example of a computer system suitable for use with the various embodiments disclosed herein. Other computer systems suitable for such use can include additional or fewer subsystems. In addition, bus 1714 is illustrative of any interconnection scheme serving to link the subsystems. Other computer architectures having different configurations of subsystems can also be utilized.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A method, comprising:
receiving sensor data of a subject, wherein the sensor data includes eye tracking data and visible light image data;
using one or more processors to automatically analyze the sensor data to determine one or more metrics; and
using the one or more metrics to determine an indicator associated with a likelihood the subject is being deceptive.

2. The method of claim 1, wherein the one or more metrics include a pupil feature, a fixation duration, or a gaze target.

3. The method of claim 1, wherein the one or more metrics include a response pause time, a blink rate, a heart rate, or a respiration rate.

4. The method of claim 1, wherein the one or more metrics are associated with one or more detected micro expressions.

5. The method of claim 4, wherein the one or more detected micro expressions include a lip press, a chin raise, a shoulder shrug, a mouth shrug, or a lip shrug.

6. The method of claim 1, further comprising:
using the one or more processors to automatically analyze the sensor data to determine one or more baseline metrics of the subject.

7. The method of claim 1, wherein the sensor data includes thermal sensor data and audio data.

8. The method of claim 7, further comprising using the one or more processors to automatically analyze the thermal sensor data to determine a respiration rate.

9. The method of claim 1, wherein the indicator is a percentage value or a Boolean value.

10. The method of claim 1, wherein the indicator is associated with a fixation heatmap user interface component, a baseline user interface component, a blink rate user interface component, a pause time user interface component, or a micro-expressions user interface component.

11. The method of claim 1, further comprising automatically detecting a behavioral cue associated with the determined one or more metrics.

12. The method of claim 11, further comprising automatically predicting an emotion response associated with the detected behavioral cue.

13. The method of claim 1, further comprising storing the one or more determined metrics and the determined indicator associated with the likelihood the subject is being deceptive in a remote data store.

14. A system, comprising:
one or more processors; and
a memory coupled to the one or more processors, wherein the memory is configured to provide the one or more processors with instructions which when executed cause the one or more processors to:
receive sensor data of a subject, wherein the sensor data includes eye tracking data and visible light image data;
automatically analyze the sensor data to determine one or more metrics; and
using the one or more metrics, determine an indicator associated with a likelihood the subject is being deceptive.

15. The system of claim 14, wherein the one or more metrics include a pupil feature, a fixation duration, a gaze target, a response pause time, a blink rate, a heart rate, or a respiration rate.

16. The system of claim 14, wherein the one or more metrics are associated with one or more detected micro expressions.

17. The system of claim 16, wherein the one or more detected micro expressions include a lip press, a chin raise, a shoulder shrug, a mouth shrug, or a lip shrug.

18. The system of claim 14, wherein the memory is further configured to provide the one or more processors with the instructions which when executed cause the one or more processors to automatically analyze the sensor data to determine one or more baseline metrics of the subject.

19. The system of claim 14, wherein the sensor data includes thermal sensor data and audio data.

20. A computer program product, the computer program product being embodied in a non-transitory computer readable storage medium and comprising computer instructions for:
receiving sensor data of a subject, wherein the sensor data includes eye tracking data and visible light image data;
using one or more processors to automatically analyze the sensor data to determine one or more metrics; and
using the one or more metrics to determine an indicator associated with a likelihood the subject is being deceptive.
